(19) United States
(12) Patent Application Publication
(10) Pub. No.: US 2021/0012892 A1
(43) Pub. Date: Jan. 14, 2021

(54) SYSTEM AND METHOD FOR SHARING MEDICAL INFORMATION, AND SYSTEM AND METHOD FOR DISPLAYING MEDICAL INFORMATION

(71) Applicant: PANASONIC INTELLECTUAL PROPERTY MANAGEMENT CO., LTD., Osaka (JP)

(72) Inventors: Kohei NAGURA, Kanagawa (JP); Yoshiki YAMAMOTO, Kanagawa (JP); Shuichi KASAI, Kanagawa (JP); Toshiki YAMAGUCHI, Kanagawa (JP); Mariko MURAKAMI, Osaka (JP)

(73) Assignee: PANASONIC INTELLECTUAL PROPERTY MANAGEMENT CO., LTD., Osaka (JP)

(21) Appl. No.: 16/958,075

(22) PCT Filed: Nov. 14, 2018
(86) PCT No.: PCT/JP2018/042165

§ 371 Date: Aug. 6, 2020

(30) Foreign Application Priority Data
Dec. 28, 2017 (JP) 2017-253177
Dec. 28, 2017 (JP) 2017-253178

(51) Int. Cl. G16H 40/67; G06F 3/14; G16H 10/60

(52) U.S. Cl. CPC G16H 40/67; G16H 10/60; G06F 3/14

(57) ABSTRACT

Provided is a system that includes an electronic clinical record server configured to manage patient attributes and main medical information records, an information sharing server configured to manage accompanying medical information records which the electronic clinical record server does not manage, and a user terminal connected to the electronic clinical record server and the information sharing server via a network and configured to enable a staff member to enter and view the main and accompanying medical information records, and where the information sharing server is configured to integrate and manage the accompanying medical information records relating to the respective services as shared medical information records based on a prescribed format, and to provide the shared medical information records to the user terminal.

### TECHNICAL FIELD

The present invention relates to a system and a method for sharing medical information, the system and the method enabling staff members in charge of different services to share medical information records of a patient; and a system and a method for displaying medical information, the system and the method displaying medical information records such that staff members in charge of different services can share medical information records.

### BACKGROUND ART

In recent years, electronic clinical record systems are widely used in hospitals, where such a system include an electronic clinical record server in which clinical records of each patient are registered so that required patient's clinical records can be viewed on a user terminal such as a personal computer.

Known such electronic clinical record systems include a system which uses a medical information template in a prescribed format in which users can enter and register medical information records as clinical records. (See Patent Documents 1) Such a configuration enables users to enter patients' clinical records in an efficient manner.

Another known technology provides a device configured to show whether or not there are unread test results for a patient, which have not yet unread by a staff, by making a mark blink on the listing of patient's data or automatically indicating the patient's medical treatment history information. (See Patent Document 2) Such a configuration ensures that a staff can recognize a large number of requested test results, thereby preventing the staff from overlooking the test results.

### PRIOR ART DOCUMENT(S)

### Patent Document(S)

Patent Document 1: JP2001-118012A

Patent Document 2: JP2006-268458A

### SUMMARY OF THE INVENTION

### Task to be Accomplished by the Invention

Main medical information records are registered and managed by an electronic clinical record server. However, since the electronic clinical record server cannot manage accompanying medical information records, subsystems for staff members in charge of different services (such as medical doctors, nurses, and pharmacists) are generally used to individually manage the respective accompanying medical information records of different services. As a result, there has been a problem that staff members in charge of a certain service cannot easily view accompanying medical information records associated with other services, resulting in that medical information records cannot be fully utilized and coordination is not promoted among staff members of the different services.

One solution related to this problem is to provide a system including an information sharing server to which a staff member in charge of each medical service can register medical information so that a plurality of staff members can share the registered medical information. However, in such a system of the above-described prior art, only a common medical information template can be shared by staff members of difference services, and the technology gives no regard to how staff members in charge of different services can share the same medical information; that is, this prior art technology cannot solve the above-described problem.

In addition, a system of the second prior art only indicates that unread medical information records are present, and the technology gives no regard to how staff members in charge of different services can share the same medical information, which means that a staff member cannot quickly recognize necessary relevant medical information records with little or no error.

More specifically, in the case of a system in which staff members in charge of different services share medical information records, staff members in charge of each service can update medical information records associated with the service as needed to thereby indicate the newest medical information records. However, in this case, since many staff members view the medical information records, it is difficult to configure such a system to indicate unread medical information records for each viewer in a similar manner to the above-described prior art. Moreover, even if the system is configured to indicate the newest information for each item separately, very old information records for some items, which have been not updated for a long time, can be indicated as newest records, which are likely to be inappropriate records, which do not represent the current conditions of a patient. As a result, a staff member may wrongly consider the indicated old records as the current conditions of the patient.

The present invention has been made in view of such problems of the prior art, and a primary object of the present invention is to provide a system and a method for sharing medical information, the system and the method enabling a plurality of staff members in charge of different services to share medical information records registered by staff members of each service so that the medical information records can be fully utilized and coordination can be promoted among the staff members of the different services, and to provide a system and a method for displaying medical information, the system and the method enabling staff members in charge of different service to share medical information records such that, when many staff members browse the medical information records, each member can quickly recognize necessary relevant medical information records with little or no error.

### Means to Accomplish the Task

An aspect of the present invention provides a system for sharing medical information, the system enabling staff members in charge of different services to share medical information records of a patient, the system comprising: a first server apparatus configured to manage patient attributes and main medical information records; a second server apparatus configured to manage accompanying medical information records which the first server apparatus does not manage; and a terminal device connected to the first server apparatus and the second server apparatus via a network and configured to enable a staff member to enter and view the main and accompanying medical information records, wherein the second server apparatus is configured to integrate and manage the accompanying medical information records relating to the respective services as shared medical information records based on a prescribed format, and to provide the shared medical information records to the terminal device.

Another aspect of the present invention provides a method for sharing medical information, the method enabling staff members in charge of different services to share medical information records of a patient, the method comprising: a first server apparatus managing patient attributes and main medical information records; a second server apparatus managing accompanying medical information records which the first server apparatus does not manage; and a terminal device enabling a staff member to enter and view the main and accompanying medical information records, the terminal device being connected to the second server apparatus via a network, wherein the second server apparatus is configured to integrate and manage the accompanying medical information records relating to the respective services as shared medical information records based on a prescribed format, and to provide the shared medical information records to the terminal device.

Yet another aspect of the present invention provides a system for displaying medical information, the system displaying medical information records such that staff members in charge of different services can share medical information records, the system comprising: a server apparatus configured to manage the medical information records such that the staff members can share the medical information records; and a terminal device connected to the server apparatus via a network and configured to enable a staff member to enter and view the medical information records, wherein the server apparatus is configured to determine whether or not to add a prescribed highlighting visual effect to a medical information record displayed on the terminal device depending on freshness of the record, the freshness being determined by an update date and time of the record.

Yet another aspect of the present invention provides a method for displaying medical information, the method displaying medical information records such that staff members in charge of different services can share medical information records, the method comprising: a server apparatus managing the medical information records such that the staff members can share the medical information records; and a terminal device enabling a staff member to enter and view the medical information records, the terminal device being connected to the server apparatus via a network, wherein the server apparatus is configured to determine whether or not to add a prescribed highlighting visual effect to a medical information record displayed on the terminal device depending on freshness of the record, the freshness being determined by an update date and time of the record.

### Effect of the Invention

According to the present invention, since a plurality of staff members in charge of different services (functions) are allowed to share medical information records registered by any staff member of each service, the registered medical information can be fully utilized and coordination can be promoted among the staff members of the different services. Moreover, since a highlighting visual effect is used for some displayed medical information records depending on freshness of the records, staff members can easily recognize freshness of medical information records. As a result, each staff member can quickly recognize necessary relevant medical information records with little or no error.

### BRIEF DESCRIPTION OF THE DRAWINGS

FIG. 1 is a diagram showing a general configuration of a system for sharing medical information according to a first embodiment of the present invention;

FIG. 2 is a block diagram showing schematic configurations of an information sharing server 2, a user terminal 3, and an administrator terminal 4 according to the first embodiment;

FIG. 3 is an explanatory diagram showing transitions of screens displayed on the user terminal 3 when a user browses and performs entry operations on a main sheet according to the first embodiment;

FIG. 4 is an explanatory diagram showing a login screen displayed on the user terminal 3 according to the first embodiment;

FIG. 5 is an explanatory diagram showing a patient search screen displayed on the user terminal 3 according to the first embodiment;

FIG. 6 is an explanatory diagram showing a main screen of electronic clinical records displayed on the user terminal 3 according to the first embodiment;

FIG. 7 is an explanatory diagram showing a main sheet browsing screen displayed on the user terminal 3 according to the first embodiment;

FIG. 8 is an explanatory diagram showing an itemized history record browsing screen displayed on the user terminal 3 according to the first embodiment;

FIG. 9 is an explanatory diagram showing a main sheet entry screen displayed on the user terminal 3 according to the first embodiment;

FIG. 10 is an explanatory diagram showing transitions of screens displayed on the user terminal 3 when a user browses and performs entry operations on a screening-sheet according to the first embodiment;

FIG. 11 is an explanatory diagram showing a screening sheet menu screen displayed on the user terminal 3 according to the first embodiment;

FIG. 12 is an explanatory diagram showing a screening sheet selection screen displayed on the user terminal 3 according to the first embodiment;

FIG. 13 is an explanatory diagram showing a screening sheet browsing screen displayed on the user terminal 3 according to the first embodiment;

FIG. 14 is an explanatory diagram showing a screening sheet entry screen displayed on the user terminal 3 according to the first embodiment;

FIG. 15 is an explanatory diagram showing transitions of screens displayed on the administrator terminal 4 when a user performs edit operations on the main sheet, a sub-sheet, and a screening sheet according to the first embodiment;

FIG. 16 is an explanatory diagram showing a main sheet edit screen displayed on the administrator terminal 4 according to the first embodiment;

FIG. 17 is an explanatory diagram showing a cell edit screen displayed on the administrator terminal 4 according to the first embodiment;

FIG. 18 is a sequence diagram of an operation procedure of the system when a user performs the entry operations on the main sheet according to the first embodiment;

FIG. 19 is a sequence diagram of an operation procedure of the system when a user performs the edit operations on the main sheet according to the first embodiment;

FIG. 20 is a diagram showing conditions required for adding background color highlights on a main sheet in a system for displaying medical information according to a second embodiment of the present invention;

FIG. 21 is an explanatory diagram showing a main sheet browsing screen displayed on the user terminal 3 according to the second embodiment;

FIG. 22 is an explanatory diagram showing a screening sheet browsing screen displayed on the user terminal 3 according to the second embodiment;

FIG. 23 is an explanatory diagram showing a highlight settings screen displayed on the administrator terminal 4 according to the second embodiment;

FIG. 24 is a sequence diagram of an operation procedure of the system when a user views the main sheet according to the second embodiment; and

FIG. 25 is a flow diagram of an operation procedure of a web server 5 when the web server generates a main sheet browsing screen according to the second embodiment.

### DESCRIPTION OF THE PREFERRED EMBODIMENT(S)

A first aspect of the present invention made to achieve the above-described object is a system for sharing medical information, the system enabling staff members in charge of different services to share medical information records of a patient, the system comprising: a first server apparatus configured to manage patient attributes and main medical information records; a second server apparatus configured to manage accompanying medical information records which the first server apparatus does not manage; and a terminal device connected to the first server apparatus and the second server apparatus via a network and configured to enable a staff member to enter and view the main and accompanying medical information records, wherein the second server apparatus is configured to integrate and manage the accompanying medical information records relating to the respective services as shared medical information records based on a prescribed format, and to provide the shared medical information records to the terminal device.

In this configuration, since a plurality of staff members in charge of different services (functions) are allowed to share medical information records registered by any staff member of each service, the registered medical information can be fully utilized and coordination can be promoted among the staff members of the different services.

A second aspect of the present invention is the system of the first aspect, wherein the second server apparatus is configured to acquire at least a partial data set of the patient attributes and the main medical information records and manage the acquired partial data set in association with the accompanying medical information records.

This configuration enables staff members to easily view patient attributes and main medical information records along with shared medical information records.

A third aspect of the present invention is the system of the first aspect, wherein the second server apparatus is configured such that, when a staff member enters the accompanying medical information records, the second server apparatus acquires a data set of an ID of the staff member and/or recording date and time, and manage the acquired data set in association with the shared medical information records.

This configuration can eliminate the need for a staff member to enter their ID data (such as name) and recording date and time data. The ID data of a staff member can be acquired from login information.

A fourth aspect of the present invention is the system of the first aspect, wherein the second server apparatus is configured to change a configuration of the prescribed format in response to an edit operation performed by a staff member.

In this configuration, since a configuration of the prescribed format can be changed as necessary, the system becomes more convenient.

A fifth aspect of the present invention is the system of the fourth aspect, wherein the second server apparatus is configured to manage accompanying medical information records in association with the shared medical information records.

This configuration enables a staff member to easily enter and view accompanying medical information records in association with the shared medical information records.

A sixth aspect of the present invention is the system of the fifth aspect, wherein the second server apparatus is configured to extract selected records of the shared medical information records in response to a selection operation performed by a staff member, manage the extracted records as digest information, and provide the digest information to the terminal device.

This configuration enables the system to present, to a staff member, digest information comprised of only limited information necessary for the staff member.

A seventh aspect of the present invention is a method for sharing medical information, the method enabling staff members in charge of different services to share medical information records of a patient, the method comprising: a first server apparatus managing patient attributes and main medical information records; a second server apparatus managing accompanying medical information records which the first server apparatus does not manage; and a terminal device enabling a staff member to enter and view the main and accompanying medical information records, the terminal device being connected to the second server apparatus via a network, wherein the second server apparatus is configured to integrate and manage the accompanying medical information records relating to the respective services as shared medical information records based on a prescribed format, and to provide the shared medical information records to the terminal device.

In this configuration, since a plurality of staff members in charge of different services (functions) are allowed to share medical information records registered by any staff member of each service, the registered medical information can be fully utilized and coordination can be promoted among the staff members of the different services in the same manner as the first aspect of the present invention.

An eighth aspect of the present invention is a system for displaying medical information, the system displaying medical information records such that staff members in charge of different services can share medical information records, the system comprising: a server apparatus configured to manage the medical information records such that the staff members can share the medical information records; and a terminal device connected to the server apparatus via a network and configured to enable a staff member to enter and view the medical information records, wherein the server apparatus is configured to determine whether or not to add a prescribed highlighting visual effect to a medical information record displayed on the terminal device depending on freshness of the record, the freshness being determined by an update date and time of the record.

In this configuration, since a highlighting visual effect is used for some displayed medical information records depending on freshness of the record, each staff member can quickly recognize necessary relevant medical information records with little or no error.

A ninth aspect of the present invention is the system of the eighth aspect, wherein the server apparatus determines the freshness of the medical information record based on whether or not the medical information record has updated within a prescribed period of time from a reference date and time, the reference date and time being specified for when the staff member browses the medical information record.

This configuration enables proper determination of freshness of a medical information record.

A tenth aspect of the present invention is the system of the ninth aspect, wherein the server apparatus is configured such that, when the update date and time of the medical information record is within a prescribed last period of time, the server apparatus adds the highlighting visual effect to the medical information record displayed on the terminal device to thereby indicate that the medical information record is a latest record.

This configuration enables staff members to quickly recognize which information has been updated recently.

An eleventh aspect of the present invention is the system of the ninth aspect, wherein the server apparatus is configured such that, when the update date and time of the medical information record is not within a prescribed reliable period of time, the server apparatus adds the highlighting visual effect to the medical information record displayed on the terminal device to thereby indicate that the medical information record is a less reliable record.

This configuration enables staff members to quickly recognize which information is not reliable. As a result, the system can call staff members' attention to such unreliable information to thereby prevent their misapprehension of the current conditions of a patient.

A twelfth aspect of the present invention is the system of the eighth aspect, wherein the server apparatus adds the highlighting visual effect to a target medical information record so that the target medical information record is displayed in a distinguishable manner from the other medical information records displayed on the terminal device, wherein the highlighting visual effect is achieved by using at least one of a background color of a display area of the target medical information record, a background pattern of the display area, a color of characters representing the target medical information record, a thickness of the characters, a decoration for the characters, and a graphic form displayed near the target medical information record.

This configuration enables staff members to easily recognize medical information to which a highlighting visual effect is added.

A thirteenth aspect of the present invention is the system of the eighth aspect, wherein each medical information record includes multiple items, and the server apparatus updates each item of the medical information record in response to entry of a corresponding item record by a staff member, and wherein, when the latest item records of the respective items are displayed on the terminal device, the server apparatus determines whether or not to add the highlighting visual effect to each item record displayed on the terminal device depending on freshness of the item record, the freshness being determined by an update date and time of the item record.

In this configuration, even in cases where the system indicates the newest information for each item separately and thus degrees of freshness of the information vary for respective items, since a highlighting visual effect is added to displayed medical information records depending on freshness of the records, staff members can easily recognize degrees of freshness of the medical records in the respective items.

Embodiments of the present invention will be described below with reference to the drawings.

### First Embodiment

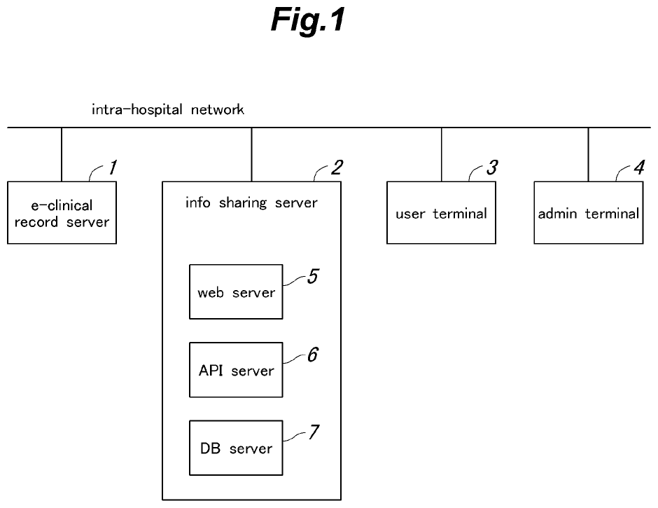

FIG. 1 is a diagram showing a general configuration of a system for sharing medical information according to a first embodiment of the present invention.

The system for sharing medical information enables staff members in charge of different services in a hospital (staff members responsible for different tasks) to share medical information records, and the system includes an electronic clinical record server 1 (first server apparatus), an information sharing server 2 (second server apparatus), a user terminal 3 (terminal device), and an administrator terminal 4 (terminal device).

All of the electronic clinical record server 1, the information sharing server 2, the user terminal 3, and the administrator terminal 4 are connected to each other via an intrahospital network.

The electronic clinical record server 1 manages clinical records of respective patients (patient attributes and main medical information records).

The information sharing server 2 manages accompanying medical information records which the electronic clinical record server 1 does not manage. The information sharing server 2 includes a web server 5 (web server) for providing medical information to user terminals 3, an API server 6 (application server) configured to perform processing operations required for managing medical information records, and a DB server 7 (database server) for storing medical information records and various settings information.

The information sharing server 2 is configured to integrate and manage accompanying medical information records associated with each of the different services as shared medical information records based on a prescribed format, and to provide the shared medical information records to the user terminal 3. Specifically, the information sharing server 2 is configured to integrate (aggregate) medical information records associated with each of the different services in a main sheet in the prescribed format, and manage the medical information records in the main sheet. The main sheet includes shared medical information tables so that staff members of each of the different services can enter corresponding medical information records into one or more of the medical information tables related to their service. In the present embodiment, the information sharing server 2 acquires part of clinical records of patients from the electronic clinical record server 1 and enters the acquired records into the main sheet. With the function of this main sheet, the medical information can be shared among staff members in charge of the different services.

In addition, the information sharing server 2 manages supplemental medical information records as a form of a sub-sheet attached to the main sheet, the supplemental medical information records (attached information records) being associated with specific items of the main sheet. The sub-sheet may be a data file generated by application software such as spread sheet software. With the function of this sub-sheet, various documents prepared in the hospital can be easily registered and made viewable as one or more sub-sheets attached to the main sheet.

Moreover, the information sharing server 2 extracts medical information records from the main sheet to generate and manage a screening sheet (digest information) in which the extracted records are aggregated (integrated).

The user terminal 3 is used by staff members. A staff member can operate the user terminal 3 to enter or view records in main sheets or screening sheets. The user terminal 3 may be a personal computer, a tablet terminal or any suitable terminal device. When the user terminal 3 is a mobile terminal such as a tablet terminal, the user terminal is connected to the intrahospital network via a wireless router.

The administrator terminal 4 is used by administrators such as managers. In the present embodiment, an administrator can operate the administrator terminal 4 to edit the format of the main sheet or the screening sheet. The administrator terminal 4 may be a personal computer or any suitable terminal device.

Users may use the user terminal 3 and the administrator terminal 4 in order to access the electronic clinical record server 1 and enter or view clinical records stored in the electronic clinical record server 1.

The user terminal 3 and the administrator terminal 4 are not different from each other in their configuration as an information processing device. In this sense, when a non-administrator user uses the device to login the system, the device is a user terminal 3, while, when a user with administrator right uses the device to login the system, the device is an administrator terminal 4.

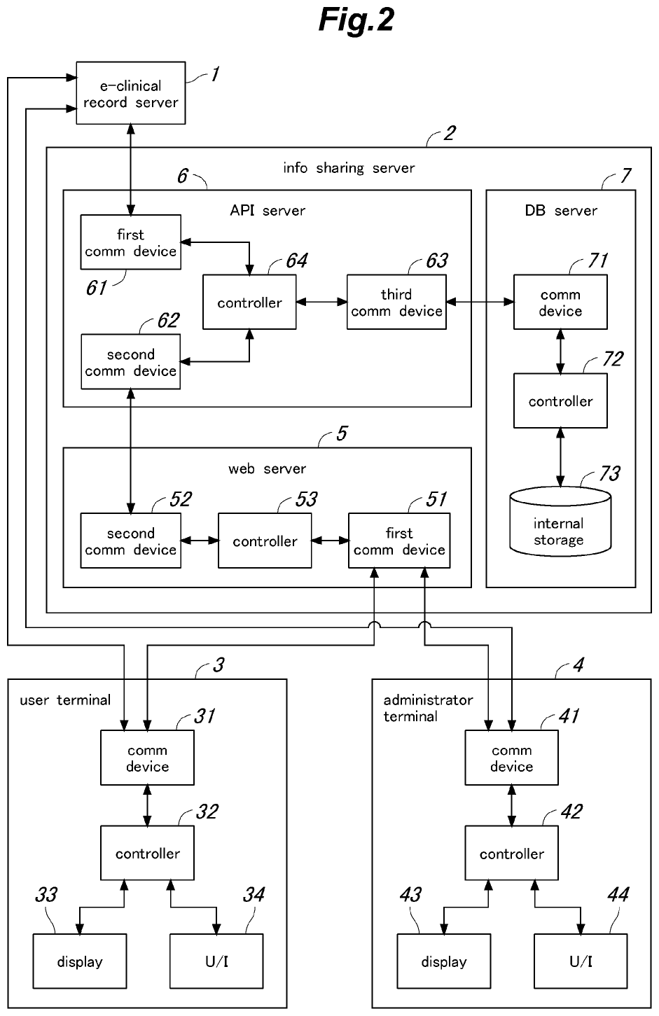

Next, schematic configurations of an information sharing server 2, a user terminal 3, and an administrator terminal 4 will be described. FIG. 2 is a block diagram showing schematic configurations of an information sharing server 2, a user terminal 3, and an administrator terminal 4 according to the first embodiment.

The information sharing server 2 includes a web server 5, an API server 6, and a DB server 7 as described above.

The web server 5 includes a first communication device 51, a second communication device 52 and a controller 53. The first communication device 51 configured to communicate with the user terminal(s) 3 and the administrator terminal(s) 4. The second communication device 52 is configured to communicate with the API server 6. The controller 53 is configured to request the API server 6 to perform necessary processing operations in response to requests from the user terminal(s) 3 and the administrator terminal(s) 4; generate a prescribed screen based on information records acquired from the API server 6; and to transmit the generated screen from the first communication device 51 to the user terminal(s) 3 and/or the administrator terminal(s) 4. In the present embodiment, for the user terminal(s) 3, the controller 53 generates screens associated with entry and browsing records in the main sheet, the sub-sheet, and the screening sheet and transmits such screens to the user terminal(s) 3. For the administrator terminal(s) 4, the controller 53 generates screens associated with editing the main sheet and the screening sheet and transmits such screens to the administrator terminal(s) 4.

The API server 6 includes a first communication device 61, a second communication device 62, a third communication device 63, and a controller 64. The first communication device 61 communicates with the electronic clinical record server 1 via the intrahospital network. The second communication device 62 communicates with the web server 5. The third communication device 63 communicates with the DB server 7. The controller 64 performs necessary processing operations such as acquiring information from the DB server 7 in response to a request from the web server 5.

The DB server 7 includes a communication device 71, a controller 72, and an internal storage 73 (storage). The communication device 71 communicates with the API server 6. The internal storage 73 stores format information including formats of the main sheet and the screening sheet, registered information records in the main sheet and the screening sheet, and other information. In response to a request from the API server 6, the controller 72 reads out information stored in the internal storage 73 and transfers the information from the communication device 71 to the API server 6. The controller 72 also stores information acquired from the API server 6 in the internal storage 73.

Each of the controllers 53, 64, and 72 is configured by a processor, and additionally includes a RAM (Random Access Memory), which is a volatile memory functioning as a work area for the processor, and a ROM (Read Only Memory), which is a non-volatile memory for storing data and control programs executable by the processor.

The web server 5, the API server 6, and the DB server 7 may be independently configured by respective information processing devices, but these servers may be independently implemented by respective software programs executed by a single information processing device.

The user terminal 3 includes a communication device 31, a controller 32, a display 33, and an operation interface 34. The communication device 31 communicates with electronic clinical record server 1 and information sharing server 2 via the intrahospital network. The display 33 is a display device such as a liquid crystal display panel, and the operation interface 34 is an input device such as a mouse and a keyboard. The controller 32 runs a web browser in order to indicate screens, which are provided from the web server 5 of the information sharing server 2, on the display 33, and the controller 32 also transmits information entered by user's operations on the operation interface 34 from the communication device 31 to the web server 5.

The administrator terminal 4 includes a communication device 41, a controller 42, a display 43, and an operation interface 44. Each unit of the administrator terminal 4 is the same as those of the user terminal 3.

Each of the controllers 32 and 42 is configured by a processor, and additionally includes a RAM, which is a volatile memory functioning as a work area for the processor, and a ROM (Read Only Memory), which is a non-volatile memory for storing data and control programs executable by the processor.

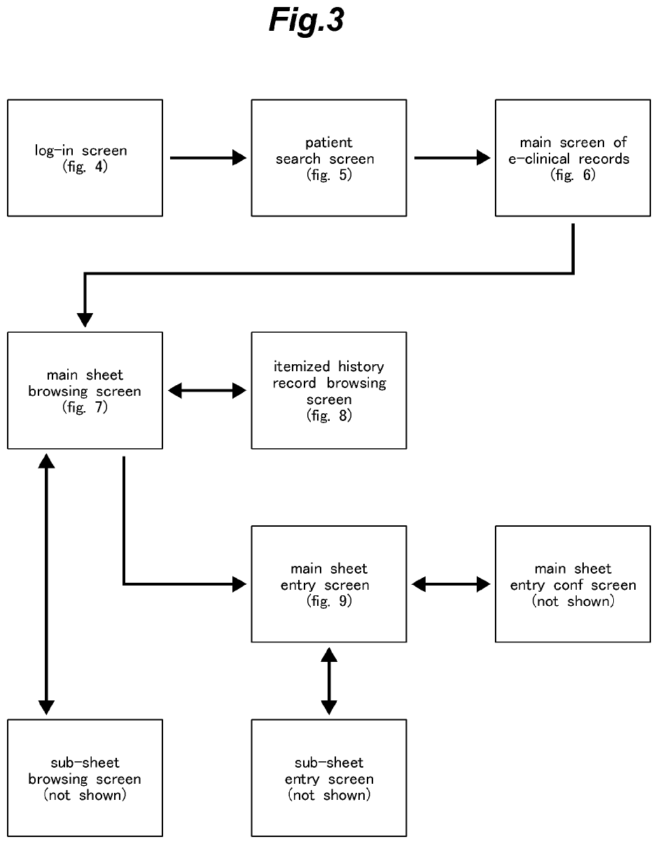

Next, screens displayed on the user terminal 3 when a user browses and performs entry operations will be described. FIG. 3 is an explanatory diagram showing transitions of screens displayed on the user terminal 3 when a user browses and performs entry operations on a main sheet.

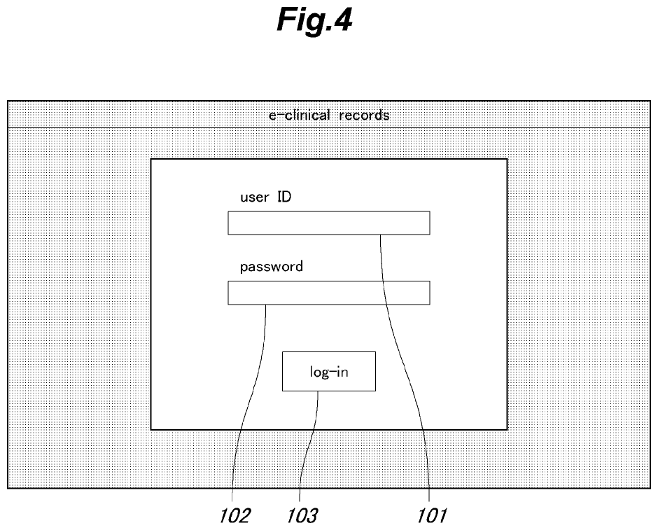

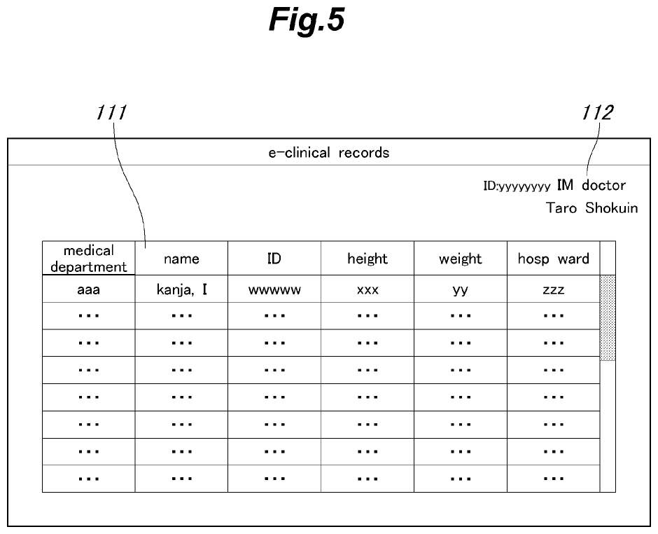

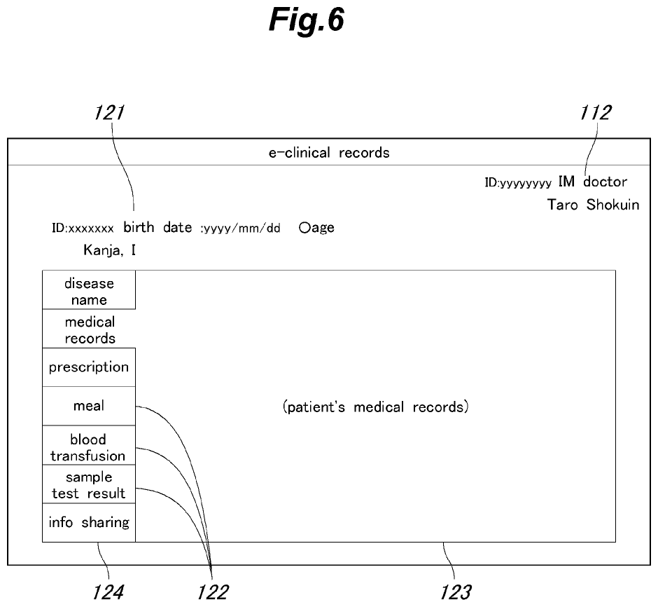

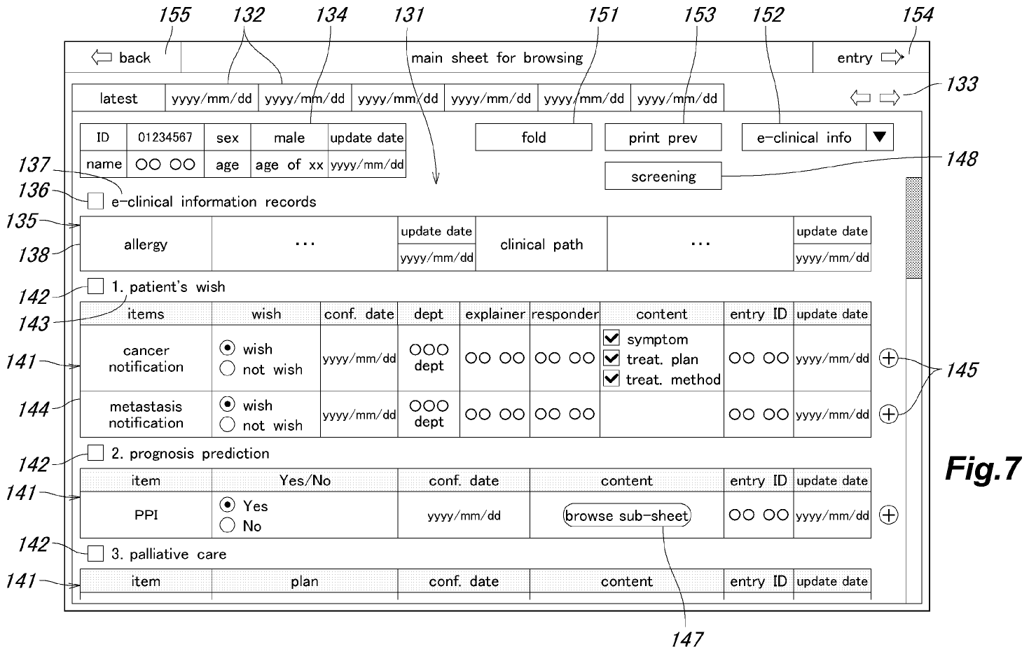

When accessing the electronic clinical record server 1, the user terminal 3 displays a login screen (see FIG. 4) for logging in to the electronic clinical record system. When a staff member performs a login operation on the login screen, the screen transitions to a patient search screen (see FIG. 5). When the staff member specifies a patient of interest on the patient search screen, the screen transitions to the main screen of the electronic clinical records (see FIG. 6). When the staff member selects “info sharing” on the main screen of the electronic clinical records, the screen transitions to the main sheet browsing screen (see FIG. 7).

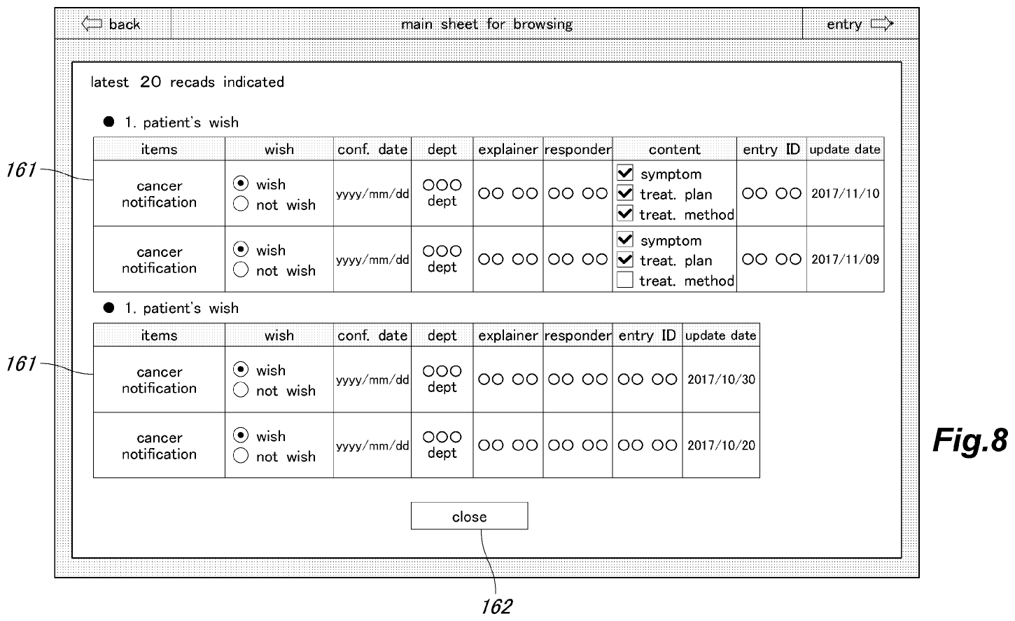

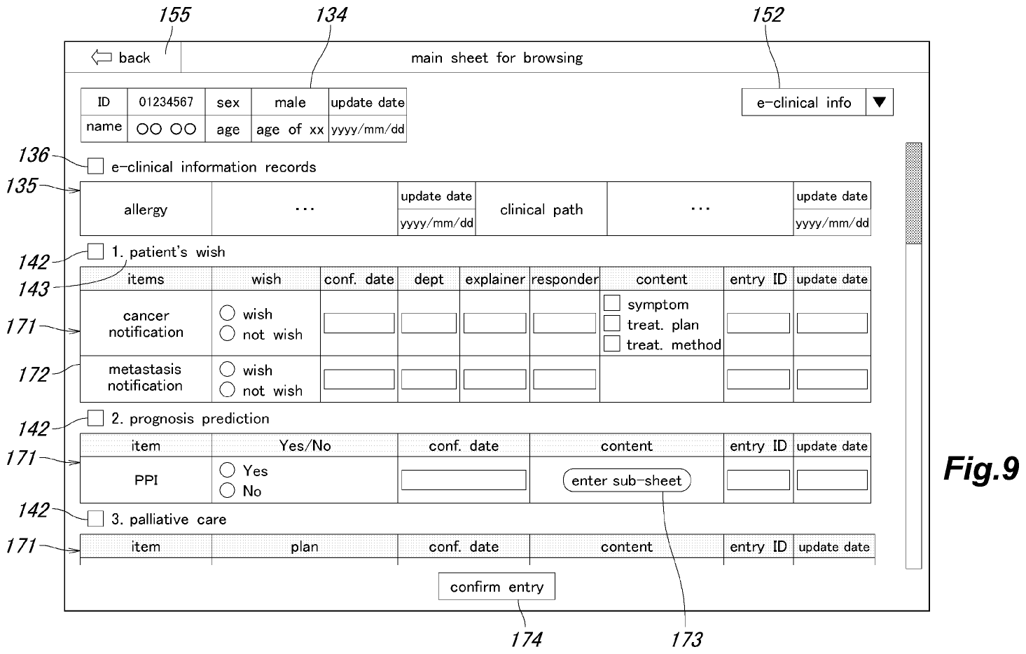

When the staff member performs a prescribed screen transition operation on the main sheet browsing screen, the screen transitions to an itemized history record browsing screen (see FIG. 8). When the staff member performs a prescribed screen transition operation on the main sheet browsing screen, the screen transitions to the main sheet entry screen (see FIG. 9). When the staff member performs a prescribed screen transition operation on the main sheet browsing screen, the screen transits to a sub-sheet browsing screen so that the staff member can browse the sub-sheet.

When the staff member performs a prescribed screen transition operation on the main sheet entry screen, the screen transitions to a main sheet entry confirmation screen. When the staff member performs a registration operation on the main sheet entry confirmation screen, the information entered into the main sheet entry screen is registered. When the staff member performs a prescribed screen transition operation on the main sheet entry screen, the screen transitions to a sub-sheet entry screen so that the staff member can register a new sub-sheet, and can update (addition or correction) the contents of the existing sub-sheet.

Next, a login screen displayed on the user terminal 3 will be described. FIG. 4 is an explanatory diagram showing the login screen displayed on the user terminal 3.

This login screen is used for logging-in to the electronic clinical record system implemented by the electronic clinical record server 1, and is displayed when the electronic clinical record server 1 is accessed by a user terminal 3.

This login screen includes a user ID entry dialog 101, a password entry dialog 102, and a “login” button 103. When a staff member enters the user ID and password and operates the “login” button 103, the electronic clinical record server 1 performs user authentication, and when the user authenticates successfully, the screen transitions to a patient search screen (see FIG. 5).

Next, a patient search screen displayed on the user terminal 3 will be described. FIG. 5 is an explanatory diagram showing the patient search screen displayed on the user terminal 3.

This patient search screen is used to search for and identify a patient of interest from a displayed list of patients, and the screen is displayed by performing the login operation on the login screen (see FIG. 4).

This patient search screen includes a patient information list indicator 111. The patient information list indicator 111 displays a patient information list; that is, a list of information for each patient (such as medical department, name, patient ID, height, weight, and hospital ward). When a staff member performs a patient selection operation on the patient information list indicator 111, the screen transitions to the main screen (see FIG. 6) of the electronic clinical records. In some cases, for the purpose of reduction in the time required to search for a specific patient, the patient search screen may include a search button (not shown) used to search the list using a search key such as name or other patient information, and a sort button (not shown) used to rearrange sets of information for respective patient.

The patient search screen also includes a user information indicator 112. The user information indicator indicates information on the logged-in staff member (such as user ID, affiliation, responsible services).

Next, a main screen of electronic clinical records displayed on the user terminal 3 will be described. FIG. 6 is an explanatory diagram showing the main screen of electronic clinical records displayed on the user terminal 3.

The main screen of the electronic clinical records displays clinical records of a patient of interest when the patient is selected in the patient search screen (see FIG. 5).

The main screen of the electronic clinical records includes a user information indicator 112 in a similar manner to the patient search screen. In addition, the main screen of the electronic clinical records includes a patient information indicator 121. The patient information indicator 121 indicates information on a patient of interest (such as patient ID, date of birth, age, and name).

The main screen of the electronic clinical records includes tabs for the respective indication items (such as disease name, medical records, prescription, meal, blood transfusion, sample test result), and a clinical record indicator 123. When a user operates one of the tabs 122 for the respective indication items, the clinical record indicator 123 indicates information relating to the item of the selected tab. The contents shown on the main screen of the electronic clinical records can vary depending on the service which a logged-in staff member is in charge of.

Also, the main screen of the electronic clinical records includes a tab 124 of “info sharing”. When a user operates the tab 124, the user logs in to an information sharing system implemented by the information sharing server 2 and transitions to a main sheet browsing screen (see FIG. 7).

Next, a main sheet browsing screen displayed on the user terminal 3 will be described. FIG. 7 is an explanatory diagram showing the main sheet browsing screen displayed on the user terminal 3.

The main sheet browsing screen is used by staff members to browse the main sheet, and is displayed when a user operates the “info sharing” tab 124 in the main screen (see FIG. 6) of the electronic clinical records.

The main sheet browsing screen includes a main sheet indicator 131, tabs 132 for respective updated dates, and left and right arrow buttons 133. When a user operates each tab 132, the main sheet of the corresponding updated date is displayed on the main sheet indicator 131. When all the tabs 132 are not simultaneously displayed on the main sheet browsing screen due to the increased updated dates, a user can scroll the tabs 132 left and right by operating the left and right arrow buttons 133, respectively.

The main sheet displayed on the main sheet indicator 131 includes a patient information indicator 134. The patient information indicator 134 indicates information on a patient of interest (such as patient ID, date of birth, age, and name). These pieces of patient information are acquired from the electronic clinical record server 1.

The main sheet also includes a clinical record indicator 135. The clinical record indicator 135 includes a check box 136, a title indicator 137, and a clinical record indicator column 138. The clinical record indicator column 138 displays clinical records of a patient of interest (such as patient ID, date of birth, age, and name). These clinical records are acquired from the electronic clinical record server 1.

The main sheet includes multiple shared medical information indicators 141. The shared medical information indicators 141 indicate information records entered (recorded) by staff members of each of the services as shared medical information so that staff member of the different services can also browse the information records. A shared medical information indicator 141 includes a check box 142, a title indicator 143, a shared medical information table 144, and a “+” button(s) 145.

The title indicator 143 indicates the title of the shared medical information table 144.

In the shared medical information table 144, row header cells (row header) indicate the names of indicated items, and column header cells (column header) indicate the names of detailed records. In the shared medical information table 144, each item (row) indicates the latest shared medical information, and each cell displays characters representing the shared medical information, radio buttons, and check boxes. Each item (row) of the shared medical information table 144 includes cells indicating an enterer (ID) and updated date. Each enterer is a staff member who entered (recorded) corresponding shared medical information records, and the updated date is the date when the shared medical information records were entered.

In the shared medical information table 144, when a sub-sheet is registered in the main sheet, a corresponding cell indicates a button 147 of “browse sub-sheet.” When a user operates the button 147, the screen transitions to a sub-sheet browsing screen.

The “+” button 145 is included in each item (row) of the shared medical information table 144. When a user operates the button 145, the screen transitions to an itemized history record browsing screen (see FIG. 8) for the corresponding item.

This main sheet browsing screen includes a “fold” button 151. When the user operates the button 151, the main sheet indicator 131 indicates only the latest information records updated within a predetermined period from a reference date which is the updated date selected by a tab 132, and the other information records are folded to be invisible. This allows staff members to quickly recognize only the latest information records.

When the user operates the “fold” button 151, which causes the button to be changed to an “expand” button, and then, when the user operates the button 151, the part of the invisible information records can be unfolded to be visible. In some cases, the initial state of the main sheet browsing screen may be set as the folded state.

The main sheet browsing screen includes a title selector 152. When a user operates the title selector 152, the title selector displays a pull-down menu (combo box) including a list of title names, and then, when the user selects one of the title names in the pull-down menu, the screen jumps to the shared medical information table 144 of the selected title. Specifically, the screen is scrolled so that the shared medical information table 144 of the selected title is displayed at the top. In this example, since the electronic clinical information is selected, the clinical record indicator column 138 is displayed at the top.

The main sheet browsing screen includes a “print preview” button 153. When a user operates the “print preview” button 153, the screen transitions to a print preview screen, which allows the main sheet to be printed out by the printer. In this case, the user can operate the check boxes 136 and 142 provided for the respective titles in order to select the titles to be printed out, thereby allowing only the information records of the selected titles to be printed out.

The main sheet browsing screen includes a “screening” button 148. When a user operates this button 148, necessary information is extracted primarily from the shared medical information registered in the main sheet so as to provide digest information; that is, extracted information which is summarized into e.g. one page.

The main sheet browsing screen includes an “entry” button 154. When a user operates the button 154, the main sheet browsing screen transitions to a main sheet entry screen (see FIG. 9).

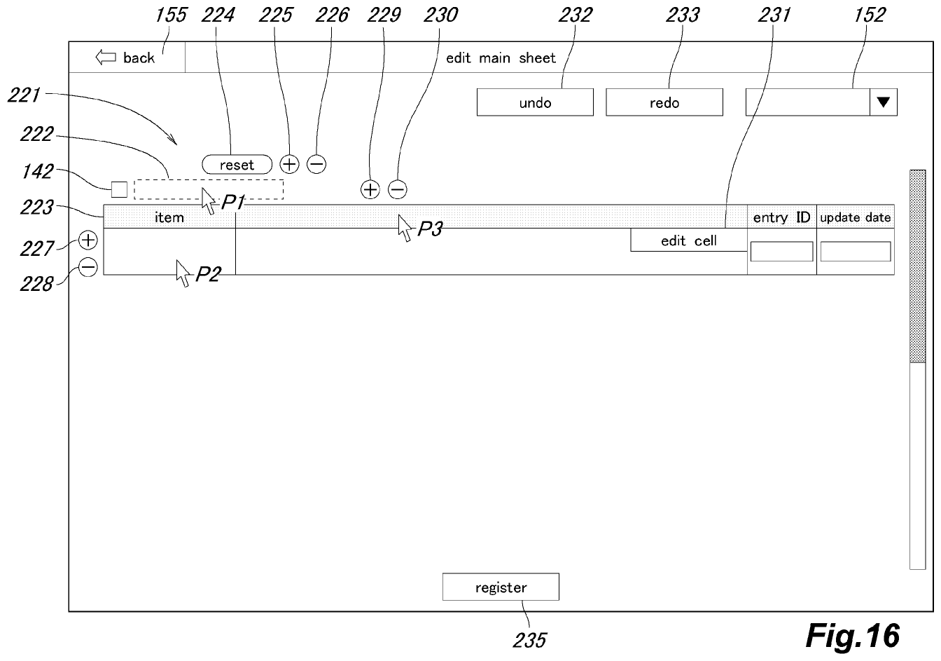

This main sheet browsing screen includes a “back” button 155. Although details of operations will not be described here, when a user operates the button 155, the screen can be changed to a settings screen for various settings (not shown). The user can perform a selection operation on the settings screen so as to transition to a main sheet edit screen (see FIG. 16) for editing the main sheet format, a sub-sheet settings screen, and a screening sheet edit screen.

Next, an itemized history record browsing screen displayed on the user terminal 3 will be described. FIG. 8 is an explanatory diagram showing the itemized history record browsing screen displayed on the user terminal 3.

This itemized history record browsing screen (dialog box) indicates a list of history information records related to the item selected on the main sheet browsing screen (see FIG. 7). When a user operates the “+” button 145 for the selected one of the items (rows) of the shared medical information table 144 on the main sheet browsing screen, the screen is changed to the itemized history record browsing screen.

The itemized history record browsing screen indicates a history information table 161. This history information table 161 indicates the shared medical information regarding the item (row) selected on the main sheet browsing screen and updated in the past, the shared medical information being listed in order from the newest one. The history information table 161 indicates a predetermined number (e.g. 20) of sets of the shared medical information from the most recently updated set. When the configuration of the shared medical information table 144 on the main sheet is changed, the history display table is divided; that is, indicated so that one history information table 161 is shown for each configuration.

When a user operates selected one of the update date cells in the history information table 161, the screen transitions to the main sheet browsing screen of the corresponding update date (see FIG. 7). Then, the screen shows the main sheet of the corresponding update date in the same manner as when a user selects one the tabs 132 for the corresponding update date.

This itemized history browsing screen includes a “close” button 162, and when a user operates the button 162, the itemized history browsing screen is closed and the screen returns to the main sheet browsing screen (see FIG. 7).

Next, a main sheet entry screen displayed on the user terminal 3 will be described. FIG. 9 is an explanatory diagram showing the main sheet entry screen displayed on the user terminal 3.

This main sheet entry screen is used by a staff member to enter necessary information into the main sheet, and is indicated when a user operates the “entry” button 154 in the main sheet browsing screen (see FIG. 7).

The main sheet entry screen includes a patient information indicator 134, a title selector 152, and a clinical record indicator 135 in a similar manner to the main sheet screen.

This main sheet entry screen includes multiple shared medical information entry fields 171. The shared medical information entry fields 171 include check boxes 142 in a non-checkable state, a title indicator 143, and a shared medical information table 172.

When a user operates each item in the shared medical information table 172, each cell of the item becomes in a ready-for-entry state, and the user can perform entry operations such as entry of texts into text boxes, selection of radio buttons or selection of check boxes.

When the user performs an edit operation on the main sheet entry screen to configure a cell so as to register a sub-sheet, an “enter sub-sheet” button 173 appears in the cell. When the user operates the button 173, the screen transitions to a sub-sheet entry screen (dialog box), which enables the user to register a new sub-sheet or update (addition or correction) of the existing sub-sheet. A new sub-sheet can also be registered from an administrator's screen.

The main sheet entry screen includes a “confirm entry” button 174. When a user operates the button 174, the screen transitions to a main sheet entry confirmation screen. The main sheet entry confirmation screen indicates the entered information in its final state. Information records of the logged-in staff member and the current date (update date) are automatically entered and indicated in an enterer ID cell and an update date cell, respectively. After confirming the entries of the main sheet on the main sheet entry confirmation screen, when the user or staff member operates a “register” button, the registered information records in the main sheet are updated by the entered information records.

In the present embodiment, the itemized history browsing screen shown in FIG. 8 indicates the history of shared medical information registered in the main sheet. Thus, when registered information records in the main sheet are updated, new shared medical information records are registered, but the previously registered shared medical information records are not deleted until a certain retention period expires.

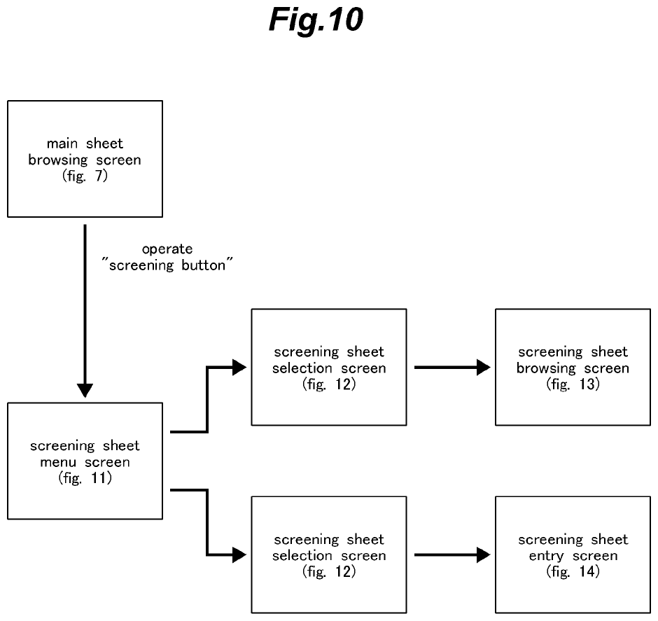

Next, a screen displayed when a user uses the user terminal 3 to browse and perform entry operations on a screening sheet, which is generated by extracting necessary information from the shared medical information registered in the main sheet, will be described. FIG. 10 is an explanatory diagram showing transitions of screens displayed on the user terminal 3 when a user browses and performs entry operations on a screening sheet.

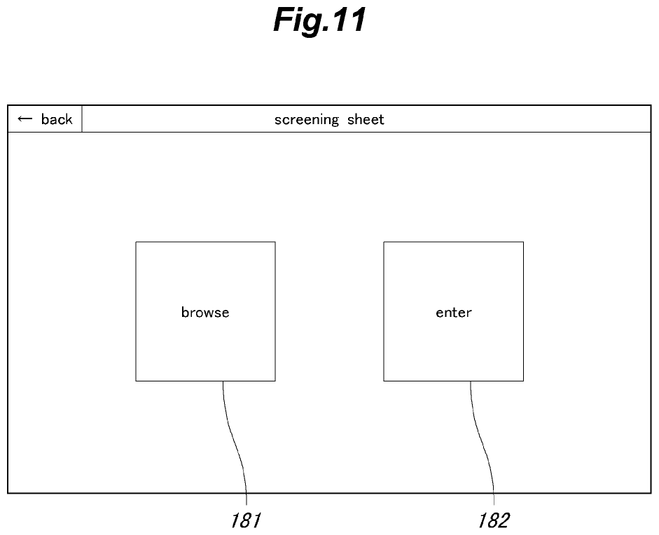

In the user terminal 3, when a staff member operates the screening button 148 on the main sheet browsing screen shown in FIG. 7, the screen transitions to a screening sheet menu screen (see FIG. 11).

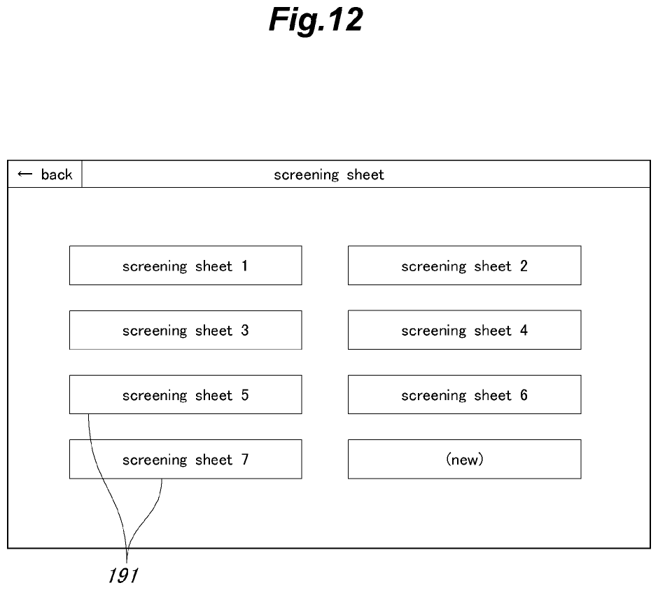

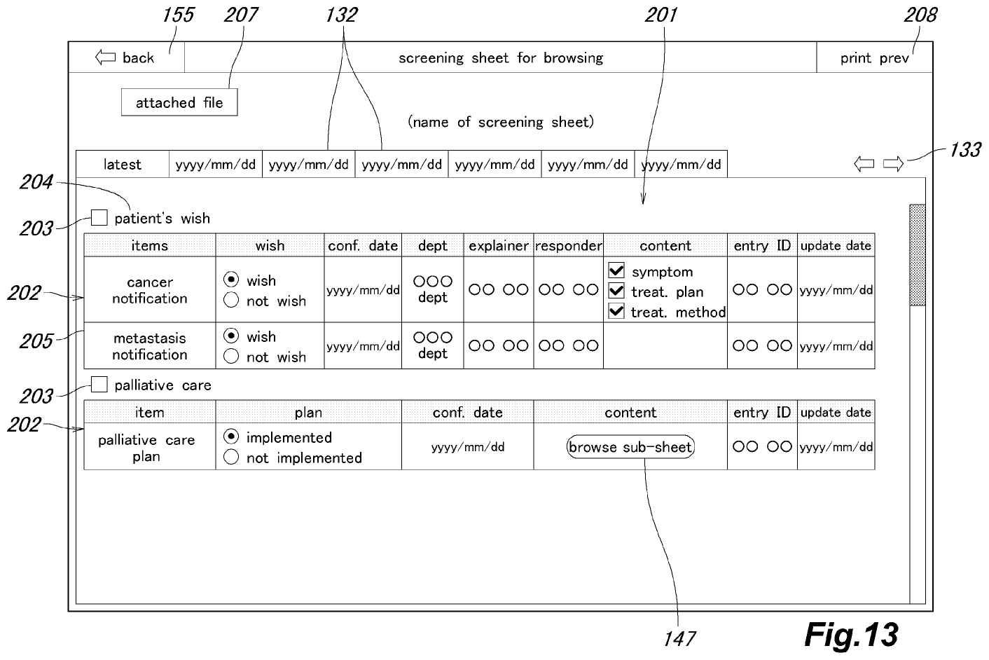

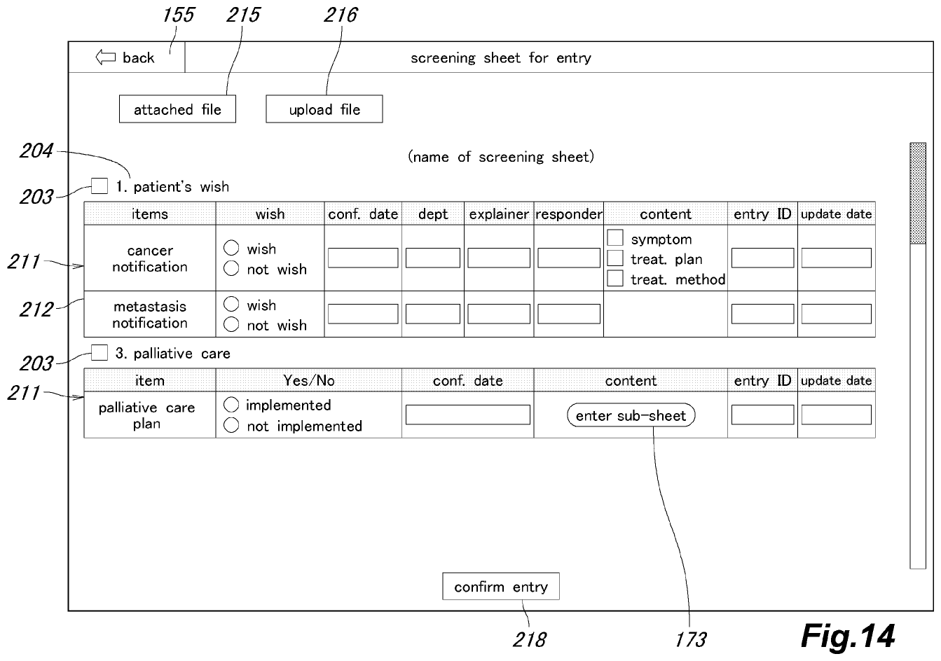

In the screening sheet menu screen, the staff member can select either browsing or entry, and when the staff member selects browsing, the screen transitions to a screening sheet selection screen (see FIG. 12). When the staff member selects screening sheet in the screening sheet selection screen, the screen transitions to a screening sheet browsing screen (see FIG. 13). When a user selects entry in the screening sheet menu screen, the screen also transitions to the screening sheet selection screen (see FIG. 12), and then further transitions to a screening sheet entry screen (see FIG. 14) for entry of information records in the latest screening sheet.

Next, the screening sheet menu screen displayed on the user terminal 3 will be described. FIG. 11 is an explanatory diagram showing the screening sheet menu screen displayed on the user terminal 3.

The screening sheet menu screen is used by a user to select either browsing or entry.

The screening sheet menu screen includes a “browse” button 181 and an “enter” button 182. When a user operates the “browse” button 181 or the “enter” button 182, the screen transitions to the screening sheet selection screen (see FIG. 12).

Next, the screening sheet selection screen displayed on the user terminal 3 will be described. FIG. 12 is an explanatory diagram showing the screening sheet selection screen displayed on the user terminal 3.

The screening sheet selection screen is used by a user to select a screening sheet browsing screen or a screening sheet entry screen, which are displayed when operating the “browse” button 181 or the “enter” button 182 on the screening sheet menu screen, respectively (see FIG. 11).

The screening sheet selection screen indicates selectors 191 for respective screening sheets, into which information records have been registered at the screening sheet edit screen, to which the settings screen for various settings transitions. When a user operates a selector 191 to select one screening sheet, the screen transitions to a screening sheet browsing screen (see FIG. 13) in which the selected screening sheet is indicated.

The selectors 191 displayed on the screening sheet selection screen includes a selector for a new screening sheet, which can be newly created and registered at the screening sheet edit screen, to which the settings screen for various settings transitions.

Next, the screening sheet browsing screen displayed on the user terminal 3 will be described. FIG. 13 is an explanatory diagram showing the screening sheet browsing screen.

The screening sheet browsing screen is used by a staff member to browse a screening sheet, which is displayed when the staff member selects one in the screening sheet selection screen (see FIG. 12).

The screening sheet browsing screen includes a screening sheet indicator 201, tabs 132 for respective update dates, and left and right arrow buttons 133. When a user operates a tab 132, the screening sheet for the corresponding updated date is indicated on the screening sheet indicator 201. The left and right arrow buttons 133 serve the same function as those in the main sheet browsing screen (see FIG. 7).

The screening sheet indicated on the screening sheet indicator 201 includes multiple shared medical information indicators 202 in a similar manner to the main sheet. Each of the shared medical information indicators 202 includes a check box 203 in a non-selectable state, a title indicator 204, and a shared medical information table 205.

Information records indicated in each cell of the shared medical information table 205 are generated from information records entered in the screening sheet entry screen and those extracted from the shared medical information table 144 in the main sheet (see FIG. 7).

The title indicated in the title indicator 204 and the names of items of the shared medical information table 205 (i.e. the names in the row header cells and the column header cells) are independently set in each of the screening sheets.

The screening sheet browsing screen includes an “attached file” button 207. When a user operates this button 207, the attached file list screen is displayed, which enables the user to select one or more of the attached files in the attached file list screen so that the user can browse the attached files.

The screening sheet browsing screen also includes a “print preview” button 208. When a user operates the button 208, the screen transitions to a print preview screen, which promotes printing out the screening sheet by using a printer.

Next, a screening sheet entry screen displayed on the user terminal 3 will be described. FIG. 14 is an explanatory diagram showing the screening sheet entry screen displayed on the user terminal 3.

The screening sheet entry screen is used by a staff member to enter necessary information records in the newest screening sheet, which is displayed when a user operates the “enter” button 182 in the screening sheet menu screen (see FIG. 11) and then selects a screening sheet in the screening sheet selection screen shown in FIG. 12.

The screening sheet entry screen includes multiple shared medical information entry fields 211. The shared medical information entry fields 211 each include a check box 203 in a non-selectable state, a title indicator 204, and a shared medical information table 212. The information in the shared medical information table 212 reflects not only newly entered information but also information extracted from the shared medical information table 144 in the main sheet (see FIG. 7).

In the shared medical information table 212, when the user operates each item, cells of the item become in a ready-for-entry state so that the user can perform entry operations on the cells such as entering text or selecting a radio button or a check box.

The screening sheet entry screen includes an “attached file” button 215. When a user operates this button 215, the screen displays a selection screen (or dialog box) indicating a list of registered attached files, and when the user selects a file in this screen, necessary application programs start to run so that the user can edit the attached file.

The screening sheet entry screen includes an “upload file” button 216. When a user operates the button 216, the screen displays a file upload screen so that the user can upload (register) one or more files stored in the user terminal 3 to the information sharing server 2.

The screening sheet entry screen includes a “confirm entry” button 218. When a user operates this button 218, the screen transitions to a screening sheet entry confirmation screen. When the user confirms the entries of the screening sheet on this screening sheet entry confirmation screen, and then operates the “register” button, the registration information records in the screening sheet are updated by the entered information records.

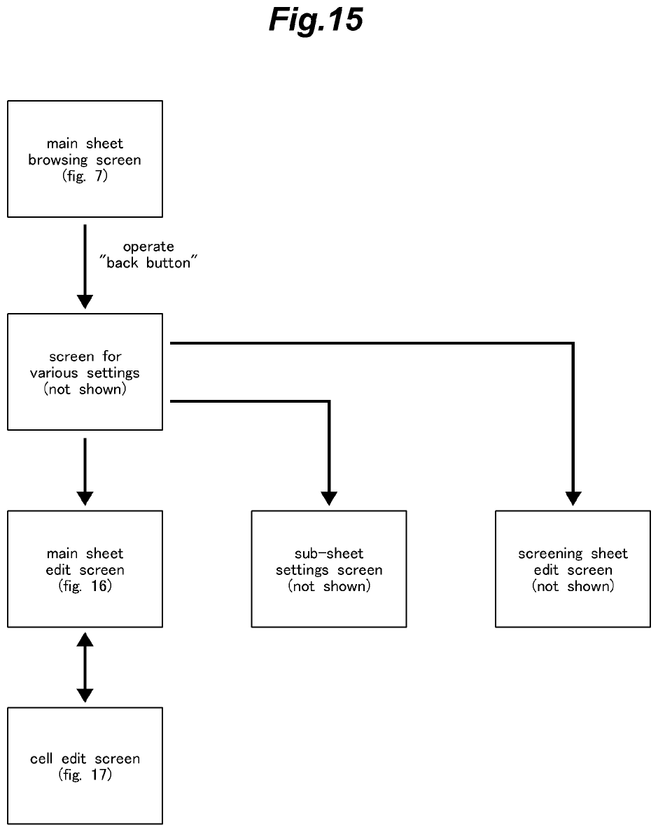

Next, screens displayed on the administrator terminal 4, to which the settings screen for various settings transitions when a user performs edit operations on the main sheet, a sub-sheet, and a screening sheet. FIG. 15 is an explanatory diagram showing transitions of screens displayed on the administrator terminal 4 when a user performs edit operations on the main sheet, a sub-sheet, and a screening sheet.

In the administrator terminal 4, the main sheet browsing screen of FIG. 7 can transition to the settings screen when a user operates a “back” button or performs another operation.

In the settings screen, a staff member can selects any of a main sheet, a sub-sheet, or a screening sheet. If the staff member selects the main sheet, the screen transitions to a main sheet edit screen (see FIG. 16), if the staff member selects the sub-sheet, the screen transitions to a sub-sheet setting screen, and if the selects the screening sheet, the screen transitions to a screening sheet edit screen.

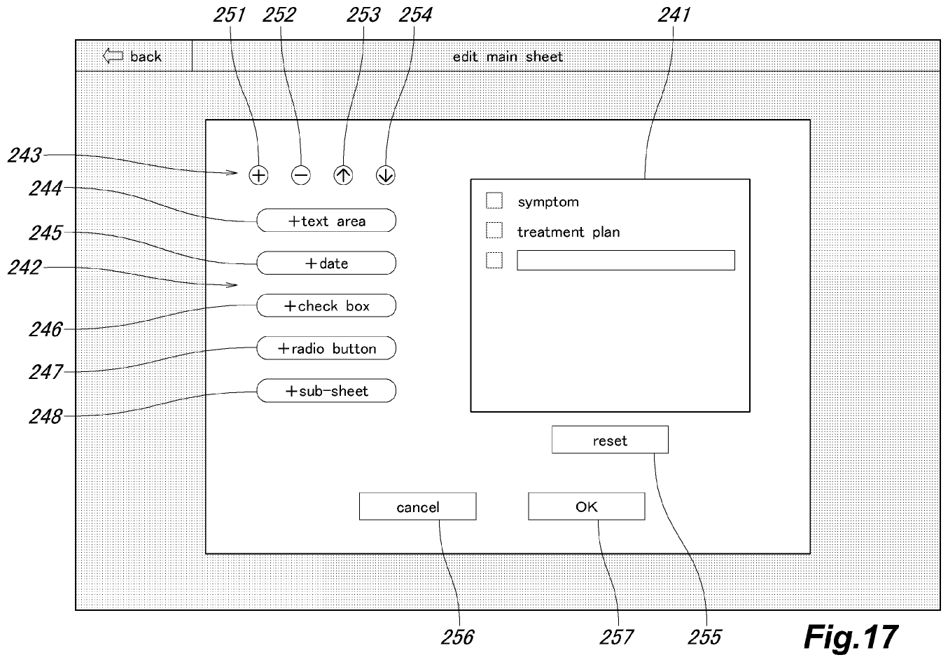

The main sheet edit screen (see FIG. 16) is used by a user to create a new main sheet or change the records in an existing main sheet. When the staff member performs a prescribed screen transition operation on the main sheet edit screen, the screen transitions to a cell edit screen (see FIG. 17) so that the user can edit cells in the tables in the main sheet.

A sub-sheet setting screen allows a user to register a sub-sheet template(s).

A screen sheet edit screen is used by a user to create a new screening sheet or change the records in an existing main sheet. In the screening sheet edit screen, a user can edit cells in the screening sheet in the same manner as the main sheet, or copy records in cells of the tables in the main sheet to those in a screening sheet. Specifically, the screen displays the original main sheet and the screening sheet to be edited, so that a user can perform a drag and drop operation to move the cells in the table in the main sheet to the screening sheet table so that the cells in the table in the main sheet are copied and pasted in the table in the screening sheet. Moreover, the administrator terminal 4 may be configured such that the screen displays check boxes for respective cells in the main sheet, thereby enabling a user to select necessary cells to be copied to the screening sheet.

Next, a main sheet edit screen displayed on the administrator terminal 4 will described. FIG. 16 is an explanatory diagram showing the main sheet edit screen displayed on the administrator terminal 4.

The main sheet edit screen is used by a user to edit the records of the main sheet. In an example shown in FIG. 16, a main sheet is newly created in which the screen indicates a basic layout of the main sheet on the main sheet edit screen. The main sheet edit screen also allows a user to edit a registered main sheet. In this case, the screen indicates the registered main sheet on the main sheet edit screen.

The basic layout of the main sheet includes only one shared medical information editor 221. The shared medical information editor 221 includes a check box 142, a title entry field 222, and a shared medical information table 223.

When a user operates the title entry field 222, the title entry field becomes in a ready-for-entry state, which allows the user to newly enter or change a character string representing the title (the name of table).

When the user performs a mouse over operation on the title entry field 222 (when the pointer is at position P1), the screen includes a reset button 224, a “+” button 225, and a “−” button 226. When the user operates the reset button 224, the records to be edited in the shared medical information editor 221 are cancelled and the screen returns to its initial state. When the user operates the “+” button 225, the shared medical information editor 221 is duplicated with the same layout. When the user operates the “−” button 226, the shared medical information editor 221 is deleted.

When the user operates the header cell at the leftmost of a row (the row header) of the shared medical information table 223, the cell becomes in a ready-for-entry state, which allows the user to newly enter or change character strings representing the item names.

When the user performs a mouse over operation on the header cell at the leftmost of a row of the shared medical information table 223 (when the pointer is at position P2), a “+” button 227 and a “−” button 228 are displayed. When the user operates the “+” button 227, the row is duplicated, and when he user operates the “−” button 228, the row is deleted.

When the user operates on the column header cells (column header) of the shared medical information table 223, the header cells becomes in a ready for entry state, which allows the user to newly enter or change character strings representing the item names.

When the user performs a mouse over operation on a column header cell of the shared medical information table 223 (when the pointer is at position P3), a “+” button 229 and a “−” button 230 are displayed. When the user operates the “+” button 229, the column is duplicated, and when he user operates the “−” button 230, the column is deleted.

A user can perform a drag and drop operation on the shared medical information editor 221 to thereby change the position of the shared medical information editor 221. A user can also perform a drag and drop operation on a row of the shared medical information table 223 to thereby switch between the positions of different rows. Furthermore, a user can perform a drag and drop operation on a column of the shared medical information table 223 to thereby exchange the positions of the different columns. Moreover, a user can perform a drag and drop operation on the line as a column boundary to thereby change the width of a column(s).

A cell edit button 231 is provided in each of the cells in which a user can enter shared medical information records in the shared medical information table 223 (that is, the cells other than the headers, cells of enterers, and cells of update dates). When a user operates the button 231, the screen transitions to a cell edit screen (see FIG. 17).

This main sheet edit screen includes an “undo” button 232 and a “redo” button 233. When a user operates the “undo” button 232, the editing operation previously made is cancelled so that the screen returns to the previous state. When a user operates the “redo” button 233, the editing operation cancelled by the “undo” button 232 is redone so that the screen is in the state before the “undo” button is pressed to cancel the operation.

The main sheet edit screen includes a title selector 152. When a user operates the title selector 152, the title selector displays a pull-down menu including a list of title names, so that the user can select one of the title names in the pull-down menu to thereby indicate the selected title for a table in the same manner as the main sheet browsing screen or the main sheet entry screen.

This main sheet edit screen includes a “register” button 235. When a user operates the button 235, the edited results are registered in the format information for the main sheet.

Next, a cell edit screen displayed on the administrator terminal 4. FIG. 17 is an explanatory diagram showing the cell edit screen displayed on the administrator terminal 4.

The cell edit screen (dialog box) is used by a user to edit cells of the shared medical information table 223 (see FIG. 16) of the main sheet, and is displayed when the user operates the cell edit button 231 displayed in each cell of the shared medical information table 223 (see FIG. 16).

The cell edit screen includes a list box 241, an item selector 242, and an item editor 243.

The list box 241 displays the edited result screen after a user operates the item selector 242 and the item editor 243.

The item selector 242 includes a text area add button 244, a date add button 245, a check box add button 246, a radio button add button 247, and a sub-sheet add button 248. When a user operates the text area add button 244, a text area for entering text is added in the cell. When a user operates the date add button 245, a date entry area is added in the cell. When the user operates the check box add button 246, a check box is added in the cell. When a user operates the radio button add button 247, a radio button is added in the cell. When a user operates the sub-sheet add button 248, a button for registering a sub-sheet is added in the cells.

The item editor 243 includes a “+” button 251, a “−” button 252, an upward arrow button 253, and a downward arrow button 254. When a user operates the “+” button 251, the selected item is duplicated. When a user operates the “−” button 252, the selected item is deleted. When a user operates the upward arrow button 253, the selected item is moved upward so that the items are switched between the selected item and the item immediately above the selected item. When a user operates the downward arrow button 254, the selected item is moved downward so that the items are switched between the selected item and the item immediately below the selected item.

This cell editor screen includes a “reset” button 255. When a user operates the button 255, the edition records displayed in the list box 241 are discarded (cancelled).

This cell edit screen includes a “cancel” button 256 and an “OK” button 257. When a user operates the “cancel” button 256, the cell edit screen is closed and the screen returns to the main sheet edit screen (see FIG. 16) and edit results are discarded. When a user operates the “OK” button 257, the cell edit screen is closed and the screen returns to the main sheet edit screen, where the edit results are reflected on the main sheet edit screen.

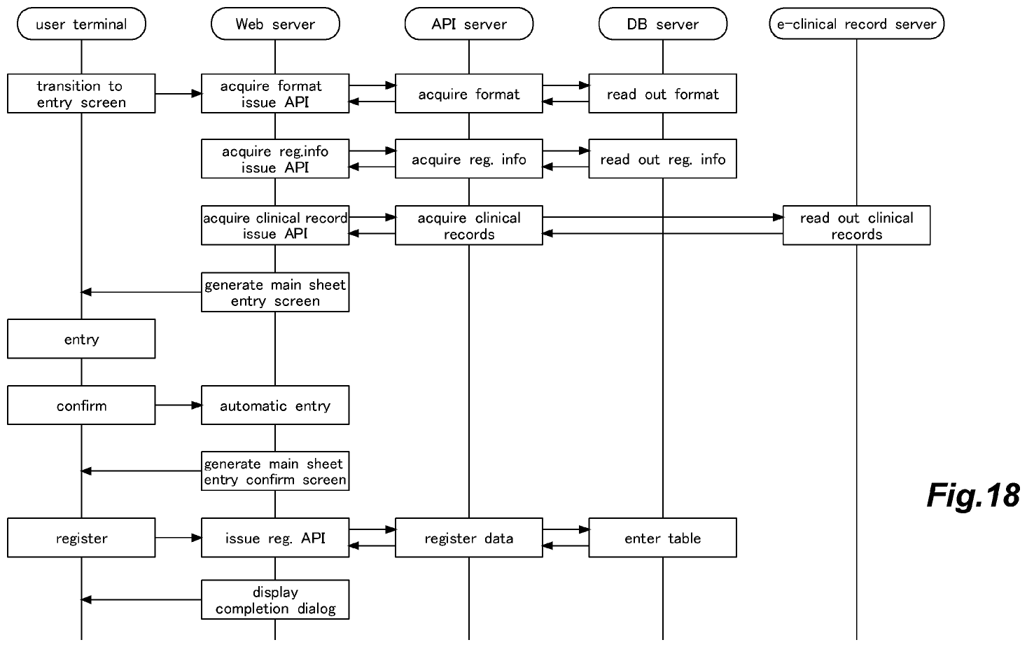

Next, a sequence diagram of an operation procedure of the system when a user performs the entry operations on the main sheet. FIG. 18 is a sequence diagram of the operation procedure of the system when a user performs the entry operations on the main sheet.

First, when a staff member operates a screen on the user terminal 3 so that the screen transitions to the main sheet entry screen, the web server 5 acquires format information for the main sheet registered in the DB server 7 via the API server 6. In addition, the web server 5 acquires information records registered in the main sheet stored in the DB server 7 via the API server 6. Moreover, the web server 5 acquires the clinical records registered in the electronic clinical record server 1 via the API server 6. Then, the web server 5 generates a main sheet entry screen (see FIG. 9) based on the main sheet format information and registered information records, and the clinical records, and displays the main sheet entry screen on the user terminal 3.

Next, in the user terminal 3, the staff member performs entry operations such as entering text to cells in the main sheet entry screen, and then confirms the entered records. Then, the web server 5 automatically enters an enterer and the update date, generates a main sheet entry confirmation screen, and causes the user terminal to display the main sheet entry confirmation screen on the user terminal 3.

Next, when, in the user terminal 3, the staff member performs an operation for registering the entered records on the main sheet entry confirmation screen, the web server 5 causes the user terminal via the API server 6 to register the information records, which have been entered in the main sheet entry screen, in the DB server 7, then causes the user terminal 3 to display a completion dialog on its screen.

Although FIG. 18 shows the operation procedure of the system when a user performs the entry operations on the main sheet, a user also performs the same entry operations in the case of the screening sheet. In addition, when a user browses the main sheet, the system performs the same operations as those shown in FIG. 18; that is, from transitioning to the main sheet entry screen until generating and displaying the main sheet entry screen.

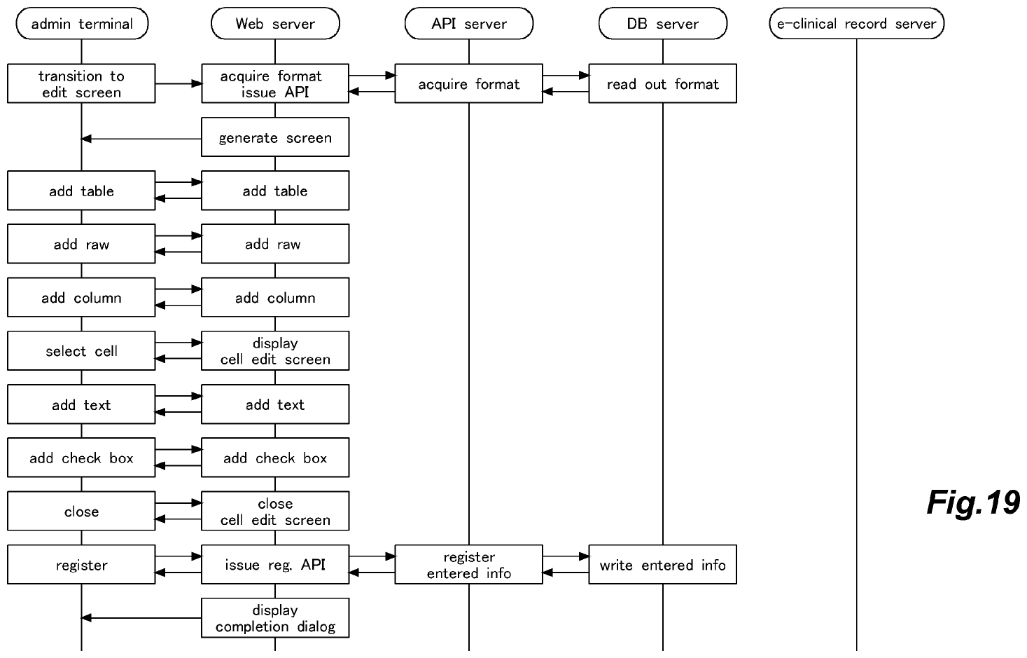

Next, an operation procedure of the system when a user performs the edit operations on the main sheet will be described. FIG. 19 is a sequence diagram of the operation procedure of the system when a user performs the edit operations on the main sheet.

First, when a staff member operates a screen on the administrator terminal 4 so that the screen transitions to the main sheet entry screen, the web server 5 acquires format information for the main sheet registered in the DB server 7 via the API server 6, (when a user creates a new main sheet, format information to be acquired is basic format information). Then, the web server 5 generates a main sheet entry screen (see FIG. 16) based on the main sheet format information and displays the main sheet entry screen on the administrator terminal 4.

Next, the staff member operates the administrator terminal 4 to perform addition of a shared medical information table and addition of rows and columns to the shared medical information table. The web server 5 performs addition of the shared medical information table and rows and columns of the table to the main sheet. When the staff member operates the administrator terminal 4 to change the records in the cell, the web server 5 causes the administrator terminal 4 to display a cell edit screen (see FIG. 17).

Next, when the staff member performs operations of adding text, a check box, and a radio button on the cell edit screen displayed on the administrator terminal 4, the web server 5 performs processing operations of adding text, a check box, and a radio button to cells. Then, when the staff member performs a screen closing operation on the administrator terminal 4, and the web server 5 causes the administrator terminal 4 to close the cell edit screen and display the main sheet edit screen.

Next, when the staff member performs an operation of registering the edit records on the administrator terminal 4, the web server 5 causes the administrator terminal 4 via the API server 6 to register information records, which have been entered in the main sheet edit screen, in the DB server 7. Then, the web server 5 causes the administrator terminal 4 to display a completion dialog.

### Second Embodiment

Next, a second embodiment of the present invention will be described. Except for what will be discussed here, this embodiment is the same as the above-described embodiment.

The system for displaying medical information according to the second embodiment is configured to display medical information records so that staff members in charge of different services in a hospital (staff members responsible for different tasks) can share medical information records, and the system includes an electronic clinical record server 1, an information sharing server 2 (server apparatus), a user terminal 3 (terminal device), and an administrator terminal 4 (terminal device) in a similar manner to the first embodiment (see FIG. 1).

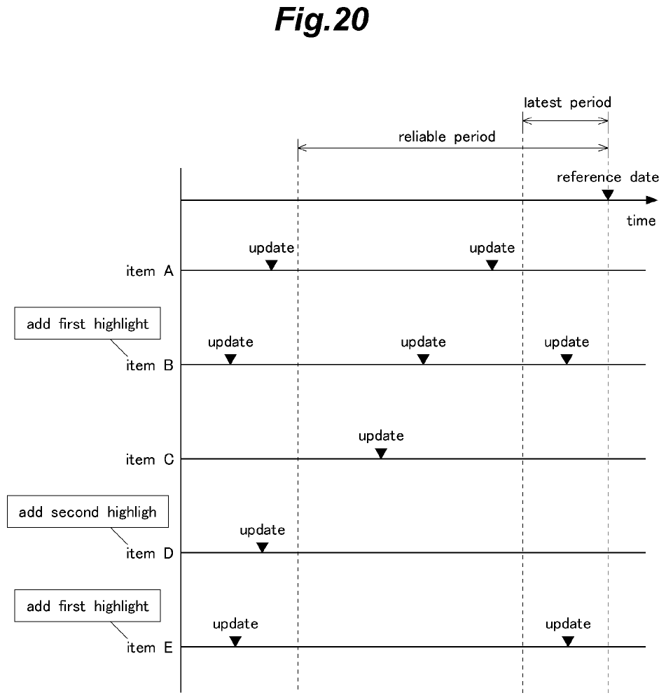

Next, background color highlights on a main sheet will be described. FIG. 20 is a diagram showing conditions required for adding background color highlights on a main sheet.

In the present embodiment, when the medical information is displayed on the main sheet of the user terminal 3, the system determines whether or not to use background color highlight (highlighting visual effect) for each item record displayed on the user terminal 3 depending on freshness of the record, the freshness being determined based on the update date and time of the record. Specifically, the system determines the freshness of medical information record based on whether or not the medical information record has updated within a prescribed period of time from a reference date and time, the reference date and time being specified for the time when the staff member browses the medical information record.

In particular, in the present embodiment, the latest period of time is set in the system such that the latest period of time is recognized as sufficiently new, and when the update date and time of the medical information record is within the latest period of time, the system uses a first background color highlight (first highlighting visual effect) for the medical information record. For example, assuming that the latest period is one day, the first background color highlight is used for a medical information record when the updated date of the medical information record is newer than one day before a reference date. In the example shown in FIG. 20, the first background color highlight is used for the items B and E.

Also, in the present embodiment, a reliable period of time set in the system such that the reliable period of time is recognized as sufficiently reliable (highly reliable), and when the update date of a medical information record is not within the reliable period of time, that is, when the reliability of medical information is low, a second background color highlight (second highlighting visual effect) is used for the medical information record. In the example shown in FIG. 20, the second background color highlight is used for the item D.

Moreover, in the present embodiment, the system may be configured such that an update date for the main sheet can be specified. In this case, if the update date is today, today is set as a reference date for the latest period of time and the reliable period of time, and if a past update date is specified, the update date is set as a reference date for the latest period of time and the reliable period of time.

Although, in the present embodiment, the freshness of medical information records is determined on a daily basis, the system may be configured to determine the freshness of medical information records on an hourly, weekly or monthly basis.

Although the background color highlights used on the main sheet have been described with reference to FIG. 20, the same background color highlights are used on the screening sheet. However, since, in the screening sheet, cells of different items (rows) on the main sheet can be copied and pasted to one item, the update dates may vary between different cells of one item, and thus highlights are not used item by item (row by row).

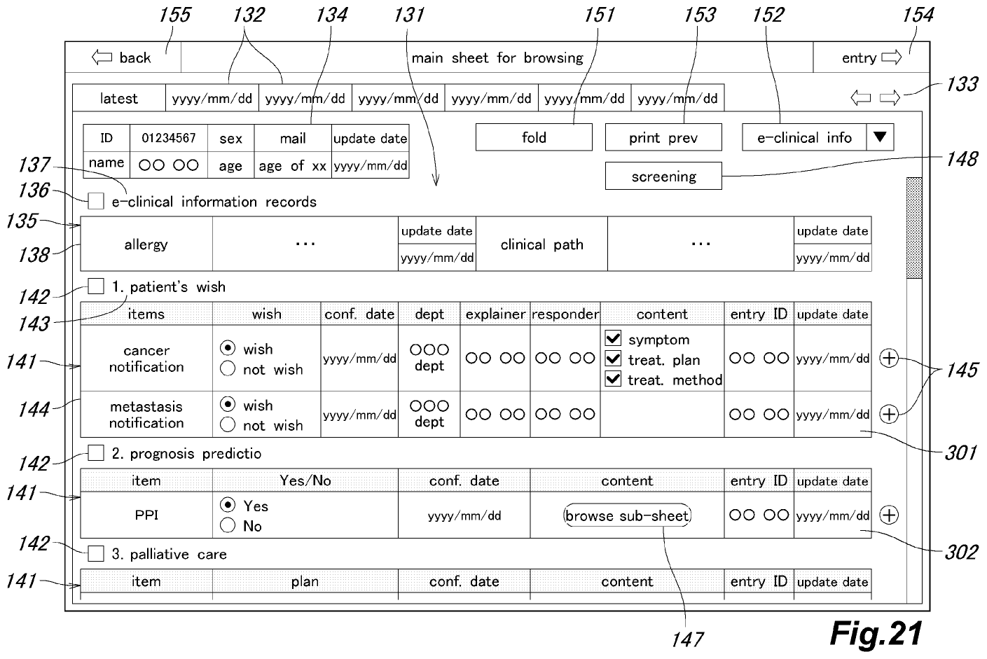

Next, a main sheet browsing screen displayed on the user terminal 3 will be described. FIG. 21 is an explanatory diagram showing the main sheet browsing screen.

On the main sheet browsing screen, background color highlights 301 and 302 are used for medical information records depending on the freshness of a medical information record, the freshness being determined based on the update date. Specifically, when the update date of a medical information record is included in the latest period of time, the first background color highlight 301 is used for the medical information record. When the update date of a medical information record is included in the reliable period time, the second background color highlight 302 is used for the medical information record.

In this example, the main sheet of the update date selected by the tab 132 is displayed on the main sheet browsing screen. When the “latest” tab 132 is selected, today is set as a reference date for the latest period of time and the reliable period, and the background color highlights 301 and 302 are used based on the reference date. In this case, for example, assuming that the latest period of time is one day, the first background color highlight 301 is used for medical information records updated today and yesterday. When the tab 132 of a past update date is selected, the update date is set as a reference date for the latest period of time and the reliable period of time, and the background color highlights 301 and 302 are used based on that reference date.

In the present embodiment, the background color highlights 301, 302, which utilize the change in the background color (filling color) of a cell (indication area for a medical information record), are used as highlighting visual effects. However, usable highlighting visual effects for cells, which are not limited to such background color highlights, may be background pattern change highlights. In other cases, highlighting visual effects may be changes in font color, font's line thickness, or other font attributes. Examples of font attributes include darken background color and brighten font color, italic, bold, underlined, and shaded. Other highlighting features include eye-catching figures (such as marks, icons) indicated near respective cells which staff members can easily notice.

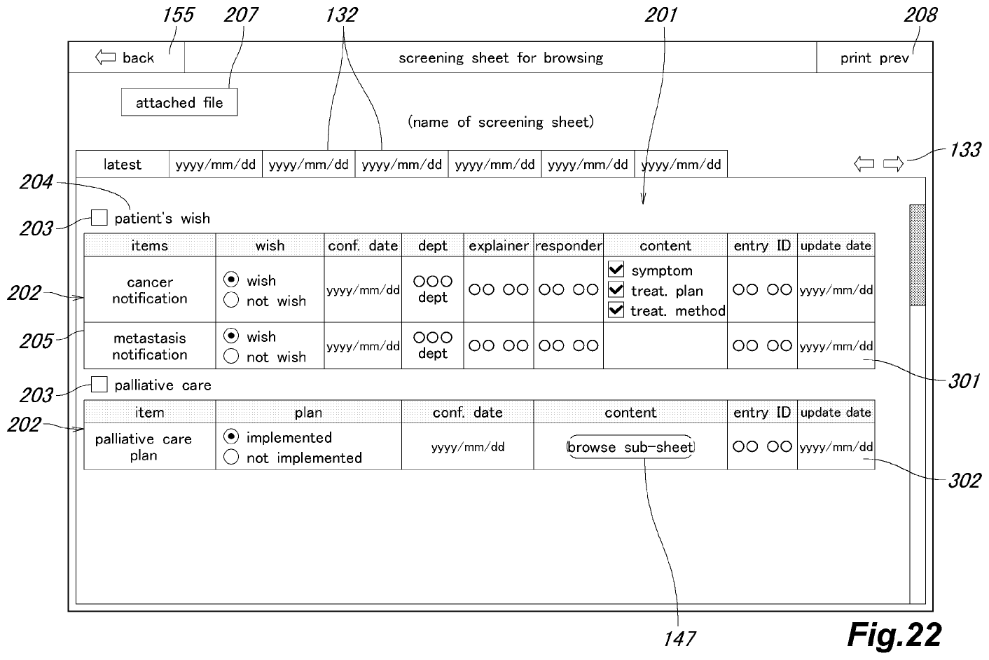

Next, a screening sheet browsing screen displayed on the user terminal 3 will be described. FIG. 22 is an explanatory diagram showing the screening sheet browsing screen displayed on the user terminal 3.

The first and second background color highlights 301 and 302 are used on the screening sheet browsing screen in a similar manner to the main sheet browsing screen.

Since, in the screening sheet, cells of different items (rows) on the main sheet can be copied and pasted to one item, the update dates may vary between different cells of one item. Thus, the background color highlights 301 and 302 are not used item by item (row by row), but used cell by cell for which the necessary condition is met.

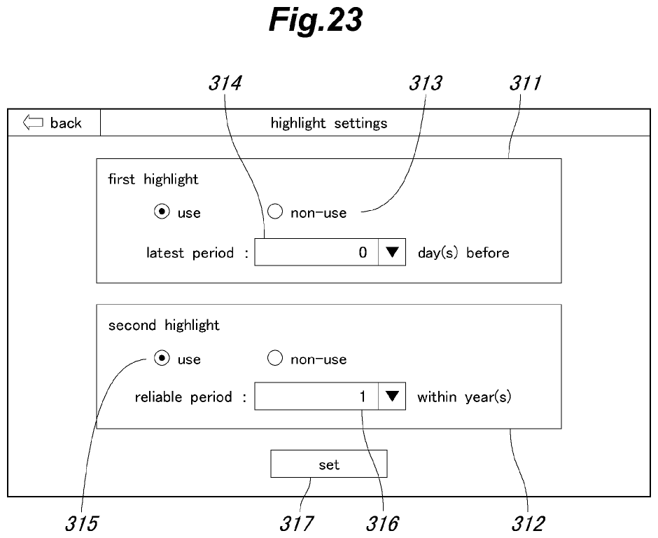

Next, a highlight settings screen displayed on the administrator terminal 4 will be described. FIG. 23 is an explanatory diagram showing the highlight settings screen displayed on the administrator terminal 4.

The highlight settings screen is used by a user to set the conditions for adding background color highlights on the main sheet and the screening sheet, and to which the settings screen for various settings transitions when a user selects highlight settings on the highlight settings screen. The conditions for adding background color highlights can be set for item by item (row by row).

The highlight settings screen includes a first background color highlight setter 311 and a second background color highlight setter 312.

The first background color highlight setter 311 a first background color highlight selector 313 and a latest period setter 314. The first background color highlight selector 313 is used by a staff member to determine whether or not the first background color highlight is used. The latest period setter 314 is used for a staff member to determine a latest period of time. In the example shown in FIG. 23, a user can select the latest period of time on a day-to-day basis by using a pulldown menu. The latest period setter 314 may be configured such that a user can select the latest period of time on an hour to hour basis or on a week to week basis.

For example, if the latest period is set to “0” day ago, the first background color highlight is used for the item (row) for records with the update date that is today; that is, a reference date, and in the screening sheet browsing screen and the main sheet browsing screen, the first background color highlight is used for information records updated on the reference date.

The second background color highlight setter 312 includes a second background color highlight selector 315 and a reliable period setter 316. The second background color highlight selector 315 is used by a staff member to determine whether or not the second background color highlight is used. The reliable period setter 316 is used for a staff member to determine a reliable period of time. In the example shown in FIG. 23, a user can select the reliable period of time on a year-to-year basis by using a pulldown menu. The reliable period setter 316 may be configured such that a user can select the reliable period of time on a day-to-day basis or on a month-to-month basis.

For example, if the reliable period is set to within “1” year, the second background color highlight is used for the item (row) for records with the update date that is before a year ago; that is, a reference date, and in the screening sheet browsing screen and the main sheet browsing screen, the second background color highlights are used for information records updated before the reference date.

The highlight settings screen also includes a “set” button 317. When a user operations the button 317, information records entered on the highlight settings screen are registered as user's settings information.

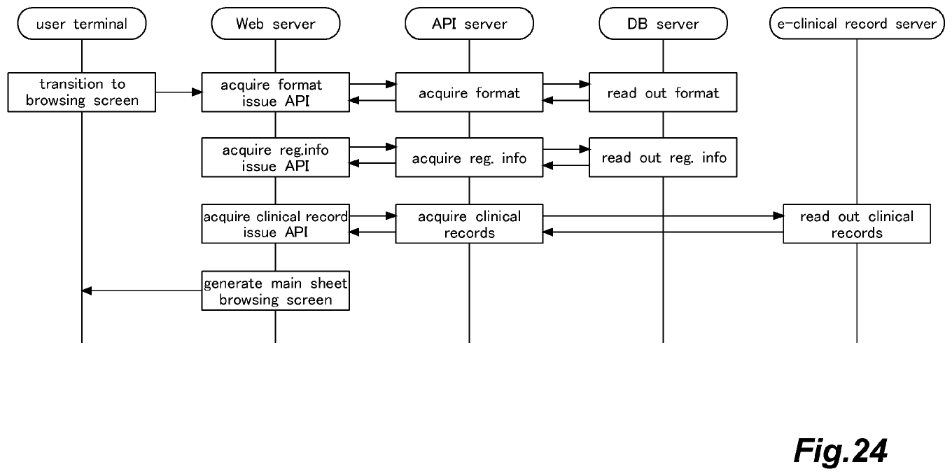

Next, an operation procedure of the system when a user browses the main sheet will be described. FIG. 24 is a sequence diagram of the operation procedure of the system when a user browses the main sheet.

First, when a staff member operates a screen on the user terminal 3 so that the screen transitions to the main sheet browsing screen, the web server 5 acquires format information for the main sheet registered in the DB server 7 via the API server 6. In addition, the web server 5 acquires information records registered in the main sheet stored in the DB server 7 via the API server 6. Moreover, the web server 5 acquires the clinical records registered in the electronic clinical record server 1 via the API server 6. Then, the web server 5 generates a main sheet browsing screen (see FIG. 21) based on the main sheet format information, registered information records, and the clinical records, and then displays the main sheet browsing screen on the user terminal 3.

Although FIG. 24 shows the operation procedure of the system when a user browses the main sheet, the system performs the same operations when a user browses the screening sheet.

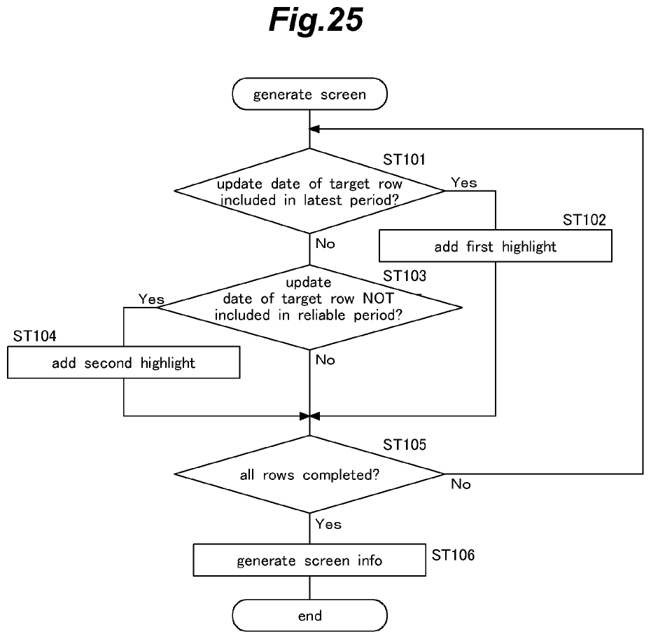

Next, an operation procedure of the web server 5 when the web server generates a main sheet browsing screen will be described. FIG. 25 is a flow diagram of the operation procedure of the web server 5 when the web server generates a main sheet browsing screen.

Upon receiving the update information from the API server 6, the web server 5 first determines whether or not the update date of a target row (item) in the shared medical information table in the main sheet is included in the latest period of time (ST101).

If the update date of the target row (item) is included in the latest period of time (Yes in ST101), the first background color highlight is set to be used for the target row (item), that is, the background color of each cell of the row is set to the color of the first background color highlight (ST102). Then, the process proceeds to ST105.

If the update date of the target row (item) is not included in the latest period of time (No in ST101), the web server 5 determines whether or not the update date of the target row (item) is included in the reliable period of time (ST103).

If the update date of the target row (item) is not included in the reliable period of time (Yes in ST103), the second background color highlight is set to be used for the target row (item), that is, the background color of each cell of the row is set to the color of the second background color highlight (ST104). Then, the process proceeds to ST105.

If the update date of the target row (item) is included in the reliable period of time (No in ST103), the process proceeds to ST105 without using either of the background color highlights.

In ST105, the web server determines whether or not the web server 5 has performed the processing operations on all the rows (items). If the web server 5 has not performed the processing operations on all the rows (items) (No in ST105), the process returns to ST101, and the web server 5 performs the processing operations on the next line (item).

If the web server 5 has performed the processing operations on all the rows (items) (Yes in ST105), the web server 5 generates a main sheet browsing screen (see FIG. 21) based on background color highlight settings information (ST106).

Although, in the example shown in FIG. 25, the web server determines use of background color highlights in the order of the first background color highlight and the second background color highlight, the first determination may be made for either of the first and second background color highlights. It should be noted that the operations regarding use of the background color highlights may be performed on the screening sheet in the same manner as that disclosed in FIG. 25 except that the operations are performed cell by cell.

While specific embodiments of the present invention are described herein for illustrative purposes, the present invention is not limited to the specific embodiments. It will be understood that various changes, substitutions, additions, and omissions may be made for elements of the embodiments without departing from the scope of the invention. In addition, elements and features of the different embodiments may be combined with each other as appropriate to yield an embodiment which is within the scope of the present invention.

For example, although, in the above-described embodiments, the system is configured such that a first server apparatus is an electronic clinical record server, a second server apparatus is an information sharing server, and the information sharing server acquires patient attributes and main medical information records for patients from the electronic clinical record server, the first server apparatus is not limited to the electronic clinical record server. For example, the first server apparatus (electronic clinical record server) may be a care management server for a care facility. In this case, the system is configured such that the care management server manages patient attributes and main caring information records for patients, and that the information sharing server manages, as shared information, accompanying caring information records which the care management server does not manage. As a result, staff members in charge of different services can share the same caring information records so that each staff member can quickly recognize necessary relevant caring information records with little or no error in the same manner as medical information records.

### INDUSTRIAL APPLICABILITY

A system and a method for sharing medical information of the present invention provides an effect of enabling staff members in charge of different medical services to share medical information records collected by staff members of each service so that the registered medical information can be fully utilized and coordination can be promoted among staff members of the different services, and are useful as a system and a method for sharing medical information, the system and the method enabling staff members in charge of different services to share medical information records of a patient. Also, a system and a method for displaying medical information of the present invention provides an effect of enabling staff members in charge of different service to share medical information records such that, when many staff members browse the medical information records, each member can quickly recognize necessary relevant medical information records with little or no error, and are useful as a system and a method for displaying medical information. the system and the method displaying medical information records such that staff members in charge of different services can share medical information records.

### Glossary

1 electronic clinical record server (first server apparatus)
2 information sharing server (second server apparatus)
3 user terminal (terminal device)
4. administrator terminal (terminal device)
301, 302 background color highlight (highlighting visual effect)

1. A system for sharing medical information, the system enabling staff members in charge of different services to share medical information records of a patient, the system comprising:
a first server apparatus configured to manage patient attributes and main medical information records;
a second server apparatus configured to manage accompanying medical information records which the first server apparatus does not manage; and
a terminal device connected to the first server apparatus and the second server apparatus via a network and configured to enable a staff member to enter and view the main and accompanying medical information records,
wherein the second server apparatus is configured to integrate and manage the accompanying medical information records relating to the respective services as shared medical information records based on a prescribed format, and to provide the shared medical information records to the terminal device.

2. The system for sharing medical information according to claim 1, wherein the second server apparatus is configured to acquire at least a partial data set of the patient attributes and the main medical information records and manage the acquired partial data set in association with the accompanying medical information records.

3. The system for sharing medical information according to claim 1, wherein the second server apparatus is configured such that, when a staff member enters the accompanying medical information records, the second server apparatus acquires a data set of an ID of the staff member and/or recording date and time, and manage the acquired data set in association with the shared medical information records.

4. The system for sharing medical information according to claim 1, wherein the second server apparatus is configured to change a configuration of the prescribed format in response to an edit operation performed by a staff member.

5. The system for sharing medical information according to claim 4, wherein the second server apparatus is configured to manage accompanying medical information records in association with the shared medical information records.

6. The system for sharing medical information according to claim 5, wherein the second server apparatus is configured to extract selected records of the shared medical information records in response to a selection operation performed by a staff member, manage the extracted records as digest information, and provide the digest information to the terminal device.

7. A method for sharing medical information, the method enabling staff members in charge of different services to share medical information records of a patient, the method comprising:
a first server apparatus managing patient attributes and main medical information records;
a second server apparatus managing accompanying medical information records which the first server apparatus does not manage; and
a terminal device enabling a staff member to enter and view the main and accompanying medical information records, the terminal device being connected to the second server apparatus via a network,
wherein the second server apparatus is configured to integrate and manage the accompanying medical information records relating to the respective services as shared medical information records based on a prescribed format, and to provide the shared medical information records to the terminal device.

8. A system for displaying medical information, the system displaying medical information records such that staff members in charge of different services can share medical information records, the system comprising:
a server apparatus configured to manage the medical information records such that the staff members can share the medical information records; and
a terminal device connected to the server apparatus via a network and configured to enable a staff member to enter and view the medical information records,
wherein the server apparatus is configured to determine whether or not to add a prescribed highlighting visual effect to a medical information record displayed on the terminal device depending on freshness of the record, the freshness being determined by an update date and time of the record.

9. The system for displaying medical information according to claim 8, wherein the server apparatus determines the freshness of the medical information record based on whether or not the medical information record has updated within a prescribed period of time from a reference date and time, the reference date and time being specified for when the staff member browses the medical information record.

10. The system for displaying medical information according to claim 9, wherein the server apparatus is configured such that, when the update date and time of the medical information record is within a prescribed last period of time, the server apparatus adds the highlighting visual effect to the medical information record displayed on the terminal device to thereby indicate that the medical information record is a latest record.

11. The system for displaying medical information according to claim 9, wherein the server apparatus is configured such that, when the update date and time of the medical information record is not within a prescribed reliable period of time, the server apparatus adds the highlighting visual effect to the medical information record displayed on the terminal device to thereby indicate that the medical information record is a less reliable record.

12. The system for displaying medical information according to claim 8, wherein the server apparatus adds the highlighting visual effect to a target medical information record so that the target medical information record is displayed in a distinguishable manner from the other medical information records displayed on the terminal device, wherein the highlighting visual effect is achieved by using at least one of a background color of a display area of the target medical information record, a background pattern of the display area, a color of characters representing the target medical information record, a thickness of the characters, a decoration for the characters, and a graphic form displayed near the target medical information record.

13. The system for displaying medical information according to claim 8, wherein each medical information record includes multiple items, and the server apparatus updates each item of the medical information record in response to entry of a corresponding item record by a staff member, and wherein, when the latest item records of the respective items are displayed on the terminal device, the server apparatus determines whether or not to add the highlighting visual effect to each item record displayed on the terminal device depending on freshness of the item record, the freshness being determined by an update date and time of the item record.

14. (canceled)
